SEQUENCE LISTING (8 sequences)

SEQ ID NO: 1 - DNA, 2520 bp, Homo Sapiens
  variation (0)...(0): Variant Y511A
atgaagaaatggagcagcacagacttgggggcagctgcggacccactccaaaaggacacctgcccagaccccctggatggagaccctaactccaggccacctccagccaagccccagctctccacggccaagagccgcacccggctctttgggaagggtgactcggaggaggctttcccggtggattgccctcacgaggaaggtgagctggactcctgcccgaccatcacagtcagccctgttatcaccatccagaggccaggagacggccccaccggtgccaggctgctgtcccaggactctgtcgccgccagcaccgagaagaccctcaggctctatgatcgcaggagtatctttgaagccgttgctcagaataactgccaggatctggagagcctgctgctcttcctgcagaagagcaagaagcacctcacagacaacgagttcaaagaccctgagacagggaagacctgtctgctgaaagccatgctcaacctgcacgacggacagaacaccaccatccccctgctcctggagatcgcgcggcaaacggacagcctgaaggagcttgtcaacgccagctacacggacagctactacaagggccagacagcactgcacatcgccatcgagagacgcaacatggccctggtgaccctcctggtggagaacggagcagacgtccaggctgcggcccatggggacttctttaagaaaaccaaagggcggcctggattctacttcggtgaactgcccctgtccctggccgcgtgcaccaaccagctgggcatcgtgaagttcctgctgcagaactcctggcagacggccgacatcagcgccagggactcggtgggcaacacggtgctgcacgccctggtggaggtggccgacaacacggccgacaacacgaagtttgtgacgagcatgtacaatgagattctgatgctgggggccaaactgcacccgacgctgaagctggaggagctcaccaacaagaagggaatgacgccgctggctctggcagctgggaccgggaagatcggggtcttggcctatattctccagcgggagatccaggagcccgagtgcaggcacctgtccaggaagttcaccgagtgggcctacgggcccgtgcactcctcgctgtacgacctgtcctgcatcgacacctgcgagaagaactcggtgctggaggtgatcgcctacagcagcagcgagacccctaatcgccacgacatgctcttggtggagccgctgaaccgactcctgcaggacaagtgggacagattcgtcaagcgcatcttctacttcaacttcctggtctactgcctgtacatgatcatcttcaccatggctgcctactacaggcccgtggatggcttgcctccctttaagatggaaaaaactggagactatttccgagttactggagagatcctgtctgtgttaggaggagtctacttctttttccgagggattcagtatttcctgcagaggcggccgtcgatgaagaccctgtttgtggacagcgccagtgagatgcttttctttctgcagtcactgttcatgctggccaccgtggtgctgtacttcagccacctcaaggagtatgtggcttccatggtattctccctggccttgggctggaccaacatgctctactacacccgcggtttccagcagatgggcatctatgccgtcatgatagagaagatgatcctgagagacctgtgccgtttcatgtttgtctacatcgtcttcttgttcgggttttccacagcggtggtgacgctgattgaagacgggaagaatgactccctgccgtctgagtccacgtcgcacaggtggcgggggcctgcctgcaggccccccgatagctcctacaacagcctgtactccacctgcctggagctgttcaagttcaccatcggcatgggcgacctggagttcactgagaactatgacttcaaggctgtcttcatcatcctgctgctggcctatgtaattctcacctacatcctcctgctcaacatgctcatcgccctcatgggtgagactgtcaacaagatcgcacaggagagcaagaacatctggaagctgcagagagccatcaccatcctggacacggagaagagcttccttaagtgcatgaggaaggccttccgctcaggcaagctgctgcaggtggggtacacacctgatggcaaggacgactaccggtggtgcttcagggtggacgaggtgaactggaccacctggaacaccaacgtgggcatcatcaacgaagacccgggcaactgtgagggcgtcaagcgcaccctgagcttctccctgcggtcaagcagagtttcaggcagacactggaagaactttgccctggtcccccttttaagagaggcaagtgctcgagataggcagtctgctcagcccgaggaagtttatctgcgacagttttcagggtctctgaagccagaggacgctgaggtcttcaagagtcctgccgcttccggggagaagtga

SEQ ID NO: 2 - protein, 839 aa, Homo Sapiens
  VARIANT (0)...(0): variant Y511A
MKKWSSTDLGAAADPLQKDTCPDPLDGDPNSRPPPAKPQLSTAKSRTRLFGKGDSEEAFPVDCPHEEGELDSCPTITVSPVITIQRPGDGPTGARLLSQDSVAASTEKTLRLYDRRSIFEAVAQNNCQDLESLLLFLQKSKKHLTDNEFKDPETGKTCLLKAMLNLHDGQNTTIPLLLEIARQTDSLKELVNASYTDSYYKGQTALHIAIERRNMALVTLLVENGADVQAAAHGDFFKKTKGRPGFYFGELPLSLAACTNQLGIVKFLLQNSWQTADISARDSVGNTVLHALVEVADNTADNTKFVTSMYNEILMLGAKLHPTLKLEELTNKKGMTPLALAAGTGKIGVLAYILQREIQEPECRHLSRKFTEWAYGPVHSSLYDLSCIDTCEKNSVLEVIAYSSSETPNRHDMLLVEPLNRLLQDKWDRFVKRIFYFNFLVYCLYMIIFTMAAYYRPVDGLPPFKMEKTGDYFRVTGEILSVLGGVYFFFRGIQYFLQRRPSMKTLFVDSASEMLFFLQSLFMLATVVLYFSHLKEYVASMVFSLALGWTNMLYYTRGFQQMGIYAVMIEKMILRDLCRFMFVYIVFLFGFSTAVVTLIEDGKNDSLPSESTSHRWRGPACRPPDSSYNSLYSTCLELFKFTIGMGDLEFTENYDFKAVFIILLLAYVILTYILLLNMLIALMGETVNKIAQESKNIWKLQRAITILDTEKSFLKCMRKAFRSGKLLQVGYTPDGKDDYRWCFRVDEVNWTTWNTNVGIINEDPGNCEGVKRTLSFSLRSSRVSGRHWKNFALVPLLREASARDRQSAQPEEVYLRQFSGSLKPEDAEVFKSPAASGEK

SEQ ID NO: 3 - DNA, 2520 bp, Homo Sapiens
  variation (0)...(0): Variant Y5111C
atgaagaaatggagcagcacagacttgggggcagctgcggacccactccaaaaggacacctgcccagaccccctggatggagaccctaactccaggccacctccagccaagccccagctctccacggccaagagccgcacccggctctttgggaagggtgactcggaggaggctttcccggtggattgccctcacgaggaaggtgagctggactcctgcccgaccatcacagtcagccctgttatcaccatccagaggccaggagacggccccaccggtgccaggctgctgtcccaggactctgtcgccgccagcaccgagaagaccctcaggctctatgatcgcaggagtatctttgaagccgttgctcagaataactgccaggatctggagagcctgctgctcttcctgcagaagagcaagaagcacctcacagacaacgagttcaaagaccctgagacagggaagacctgtctgctgaaagccatgctcaacctgcacgacggacagaacaccaccatccccctgctcctggagatcgcgcggcaaacggacagcctgaaggagcttgtcaacgccagctacacggacagctactacaagggccagacagcactgcacatcgccatcgagagacgcaacatggccctggtgaccctcctggtggagaacggagcagacgtccaggctgcggcccatggggacttctttaagaaaaccaaagggcggcctggattctacttcggtgaactgcccctgtccctggccgcgtgcaccaaccagctgggcatcgtgaagttcctgctgcagaactcctggcagacggccgacatcagcgccagggactcggtgggcaacacggtgctgcacgccctggtggaggtggccgacaacacggccgacaacacgaagtttgtgacgagcatgtacaatgagattctgatgctgggggccaaactgcacccgacgctgaagctggaggagctcaccaacaagaagggaatgacgccgctggctctggcagctgggaccgggaagatcggggtcttggcctatattctccagcgggagatccaggagcccgagtgcaggcacctgtccaggaagttcaccgagtgggcctacgggcccgtgcactcctcgctgtacgacctgtcctgcatcgacacctgcgagaagaactcggtgctggaggtgatcgcctacagcagcagcgagacccctaatcgccacgacatgctcttggtggagccgctgaaccgactcctgcaggacaagtgggacagattcgtcaagcgcatcttctacttcaacttcctggtctactgcctgtacatgatcatcttcaccatggctgcctactacaggcccgtggatggcttgcctccctttaagatggaaaaaactggagactatttccgagttactggagagatcctgtctgtgttaggaggagtctacttctttttccgagggattcagtatttcctgcagaggcggccgtcgatgaagaccctgtttgtggacagctgcagtgagatgcttttctttctgcagtcactgttcatgctggccaccgtggtgctgtacttcagccacctcaaggagtatgtggcttccatggtattctccctggccttgggctggaccaacatgctctactacacccgcggtttccagcagatgggcatctatgccgtcatgatagagaagatgatcctgagagacctgtgccgtttcatgtttgtctacatcgtcttcttgttcgggttttccacagcggtggtgacgctgattgaagacgggaagaatgactccctgccgtctgagtccacgtcgcacaggtggcgggggcctgcctgcaggccccccgatagctcctacaacagcctgtactccacctgcctggagctgttcaagttcaccatcggcatgggcgacctggagttcactgagaactatgacttcaaggctgtcttcatcatcctgctgctggcctatgtaattctcacctacatcctcctgctcaacatgctcatcgccctcatgggtgagactgtcaacaagatcgcacaggagagcaagaacatctggaagctgcagagagccatcaccatcctggacacggagaagagcttccttaagtgcatgaggaaggccttccgctcaggcaagctgctgcaggtggggtacacacctgatggcaaggacgactaccggtggtgcttcagggtggacgaggtgaactggaccacctggaacaccaacgtgggcatcatcaacgaagacccgggcaactgtgagggcgtcaagcgcaccctgagcttctccctgcggtcaagcagagtttcaggcagacactggaagaactttgccctggtcccccttttaagagaggcaagtgctcgagataggcagtctgctcagcccgaggaagtttatctgcgacagttttcagggtctctgaagccagaggacgctgaggtcttcaagagtcctgccgcttccggggagaagtga

SEQ ID NO: 4 - protein, 839 aa, Homo Sapiens
  VARIANT (0)...(0): Variant Y511C
MKKWSSTDLGAAADPLQKDTCPDPLDGDPNSRPPPAKPQLSTAKSRTRLFGKGDSEEAFPVDCPHEEGELDSCPTITVSPVITIQRPGDGPTGARLLSQDSVAASTEKTLRLYDRRSIFEAVAQNNCQDLESLLLFLQKSKKHLTDNEFKDPETGKTCLLKAMLNLHDGQNTTIPLLLEIARQTDSLKELVNASYTDSYYKGQTALHIAIERRNMALVTLLVENGADVQAAAHGDFFKKTKGRPGFYFGELPLSLAACTNQLGIVKFLLQNSWQTADISARDSVGNTVLHALVEVADNTADNTKFVTSMYNEILMLGAKLHPTLKLEELTNKKGMTPLALAAGTGKIGVLAYILQREIQEPECRHLSRKFTEWAYGPVHSSLYDLSCIDTCEKNSVLEVIAYSSSETPNRHDMLLVEPLNRLLQDKWDRFVKRIFYFNFLVYCLYMIIFTMAAYYRPVDGLPPFKMEKTGDYFRVTGEILSVLGGVYFFFRGIQYFLQRRPSMKTLFVDSCSEMLFFLQSLFMLATVVLYFSHLKEYVASMVFSLALGWTNMLYYTRGFQQMGIYAVMIEKMILRDLCRFMFVYIVFLFGFSTAVVTLIEDGKNDSLPSESTSHRWRGPACRPPDSSYNSLYSTCLELFKFTIGMGDLEFTENYDFKAVFIILLLAYVILTYILLLNMLIALMGETVNKIAQESKNIWKLQRAITILDTEKSFLKCMRKAFRSGKLLQVGYTPDGKDDYRWCFRVDEVNWTTWNTNVGIINEDPGNCEGVKRTLSFSLRSSRVSGRHWKNFALVPLLREASARDRQSAQPEEVYLRQFSGSLKPEDAEVFKSPAASGEK

SEQ ID NO: 5 - DNA, 2520 bp, Homo Sapiens
  variation (0)...(0): Variant S512Y
atgaagaaatggagcagcacagacttgggggcagctgcggacccactccaaaaggacacctgcccagaccccctggatggagaccctaactccaggccacctccagccaagccccagctctccacggccaagagccgcacccggctctttgggaagggtgactcggaggaggctttcccggtggattgccctcacgaggaaggtgagctggactcctgcccgaccatcacagtcagccctgttatcaccatccagaggccaggagacggccccaccggtgccaggctgctgtcccaggactctgtcgccgccagcaccgagaagaccctcaggctctatgatcgcaggagtatctttgaagccgttgctcagaataactgccaggatctggagagcctgctgctcttcctgcagaagagcaagaagcacctcacagacaacgagttcaaagaccctgagacagggaagacctgtctgctgaaagccatgctcaacctgcacgacggacagaacaccaccatccccctgctcctggagatcgcgcggcaaacggacagcctgaaggagcttgtcaacgccagctacacggacagctactacaagggccagacagcactgcacatcgccatcgagagacgcaacatggccctggtgaccctcctggtggagaacggagcagacgtccaggctgcggcccatggggacttctttaagaaaaccaaagggcggcctggattctacttcggtgaactgcccctgtccctggccgcgtgcaccaaccagctgggcatcgtgaagttcctgctgcagaactcctggcagacggccgacatcagcgccagggactcggtgggcaacacggtgctgcacgccctggtggaggtggccgacaacacggccgacaacacgaagtttgtgacgagcatgtacaatgagattctgatgctgggggccaaactgcacccgacgctgaagctggaggagctcaccaacaagaagggaatgacgccgctggctctggcagctgggaccgggaagatcggggtcttggcctatattctccagcgggagatccaggagcccgagtgcaggcacctgtccaggaagttcaccgagtgggcctacgggcccgtgcactcctcgctgtacgacctgtcctgcatcgacacctgcgagaagaactcggtgctggaggtgatcgcctacagcagcagcgagacccctaatcgccacgacatgctcttggtggagccgctgaaccgactcctgcaggacaagtgggacagattcgtcaagcgcatcttctacttcaacttcctggtctactgcctgtacatgatcatcttcaccatggctgcctactacaggcccgtggatggcttgcctccctttaagatggaaaaaactggagactatttccgagttactggagagatcctgtctgtgttaggaggagtctacttctttttccgagggattcagtatttcctgcagaggcggccgtcgatgaagaccctgtttgtggacagctactatgagatgcttttctttctgcagtcactgttcatgctggccaccgtggtgctgtacttcagccacctcaaggagtatgtggcttccatggtattctccctggccttgggctggaccaacatgctctactacacccgcggtttccagcagatgggcatctatgccgtcatgatagagaagatgatcctgagagacctgtgccgtttcatgtttgtctacatcgtcttcttgttcgggttttccacagcggtggtgacgctgattgaagacgggaagaatgactccctgccgtctgagtccacgtcgcacaggtggcgggggcctgcctgcaggccccccgatagctcctacaacagcctgtactccacctgcctggagctgttcaagttcaccatcggcatgggcgacctggagttcactgagaactatgacttcaaggctgtcttcatcatcctgctgctggcctatgtaattctcacctacatcctcctgctcaacatgctcatcgccctcatgggtgagactgtcaacaagatcgcacaggagagcaagaacatctggaagctgcagagagccatcaccatcctggacacggagaagagcttccttaagtgcatgaggaaggccttccgctcaggcaagctgctgcaggtggggtacacacctgatggcaaggacgactaccggtggtgcttcagggtggacgaggtgaactggaccacctggaacaccaacgtgggcatcatcaacgaagacccgggcaactgtgagggcgtcaagcgcaccctgagcttctccctgcggtcaagcagagtttcaggcagacactggaagaactttgccctggtcccccttttaagagaggcaagtgctcgagataggcagtctgctcagcccgaggaagtttatctgcgacagttttcagggtctctgaagccagaggacgctgaggtcttcaagagtcctgccgcttccggggagaagtga

SEQ ID NO: 6 - protein, 839 aa, Homo Sapiens
  VARIANT (0)...(0): Variant S512Y
MKKWSSTDLGAAADPLQKDTCPDPLDGDPNSRPPPAKPQLSTAKSRTRLFGKGDSEEAFPVDCPHEEGELDSCPTITVSPVITIQRPGDGPTGARLLSQDSVAASTEKTLRLYDRRSIFEAVAQNNCQDLESLLLFLQKSKKHLTDNEFKDPETGKTCLLKAMLNLHDGQNTTIPLLLEIARQTDSLKELVNASYTDSYYKGQTALHIAIERRNMALVTLLVENGADVQAAAHGDFFKKTKGRPGFYFGELPLSLAACTNQLGIVKFLLQNSWQTADISARDSVGNTVLHALVEVADNTADNTKFVTSMYNEILMLGAKLHPTLKLEELTNKKGMTPLALAAGTGKIGVLAYILQREIQEPECRHLSRKFTEWAYGPVHSSLYDLSCIDTCEKNSVLEVIAYSSSETPNRHDMLLVEPLNRLLQDKWDRFVKRIFYFNFLVYCLYMIIFTMAAYYRPVDGLPPFKMEKTGDYFRVTGEILSVLGGVYFFFRGIQYFLQRRPSMKTLFVDSYYEMLFFLQSLFMLATVVLYFSHLKEYVASMVFSLALGWTNMLYYTRGFQQMGIYAVMIEKMILRDLCRFMFVYIVFLFGFSTAVVTLIEDGKNDSLPSESTSHRWRGPACRPPDSSYNSLYSTCLELFKFTIGMGDLEFTENYDFKAVFIILLLAYVILTYILLLNMLIALMGETVNKIAQESKNIWKLQRAITILDTEKSFLKCMRKAFRSGKLLQVGYTPDGKDDYRWCFRVDEVNWTTWNTNVGIINEDPGNCEGVKRTLSFSLRSSRVSGRHWKNFALVPLLREASARDRQSAQPEEVYLRQFSGSLKPEDAEVFKSPAASGEK

SEQ ID NO: 7 - DNA, 2520 bp, Homo Sapiens
atgaagaaatggagcagcacagacttgggggcagctgcggacccactccaaaaggacacctgcccagaccccctggatggagaccctaactccaggccacctccagccaagccccagctctccacggccaagagccgcacccggctctttgggaagggtgactcggaggaggctttcccggtggattgccctcacgaggaaggtgagctggactcctgcccgaccatcacagtcagccctgttatcaccatccagaggccaggagacggccccaccggtgccaggctgctgtcccaggactctgtcgccgccagcaccgagaagaccctcaggctctatgatcgcaggagtatctttgaagccgttgctcagaataactgccaggatctggagagcctgctgctcttcctgcagaagagcaagaagcacctcacagacaacgagttcaaagaccctgagacagggaagacctgtctgctgaaagccatgctcaacctgcacgacggacagaacaccaccatccccctgctcctggagatcgcgcggcaaacggacagcctgaaggagcttgtcaacgccagctacacggacagctactacaagggccagacagcactgcacatcgccatcgagagacgcaacatggccctggtgaccctcctggtggagaacggagcagacgtccaggctgcggcccatggggacttctttaagaaaaccaaagggcggcctggattctacttcggtgaactgcccctgtccctggccgcgtgcaccaaccagctgggcatcgtgaagttcctgctgcagaactcctggcagacggccgacatcagcgccagggactcggtgggcaacacggtgctgcacgccctggtggaggtggccgacaacacggccgacaacacgaagtttgtgacgagcatgtacaatgagattctgatgctgggggccaaactgcacccgacgctgaagctggaggagctcaccaacaagaagggaatgacgccgctggctctggcagctgggaccgggaagatcggggtcttggcctatattctccagcgggagatccaggagcccgagtgcaggcacctgtccaggaagttcaccgagtgggcctacgggcccgtgcactcctcgctgtacgacctgtcctgcatcgacacctgcgagaagaactcggtgctggaggtgatcgcctacagcagcagcgagacccctaatcgccacgacatgctcttggtggagccgctgaaccgactcctgcaggacaagtgggacagattcgtcaagcgcatcttctacttcaacttcctggtctactgcctgtacatgatcatcttcaccatggctgcctactacaggcccgtggatggcttgcctccctttaagatggaaaaaactggagactatttccgagttactggagagatcctgtctgtgttaggaggagtctacttctttttccgagggattcagtatttcctgcagaggcggccgtcgatgaagaccctgtttgtggacagctacagtgagatgcttttctttctgcagtcactgttcatgctggccaccgtggtgctgtacttcagccacctcaaggagtatgtggcttccatggtattctccctggccttgggctggaccaacatgctctactacacccgcggtttccagcagatgggcatctatgccgtcatgatagagaagatgatcctgagagacctgtgccgtttcatgtttgtctacatcgtcttcttgttcgggttttccacagcggtggtgacgctgattgaagacgggaagaatgactccctgccgtctgagtccacgtcgcacaggtggcgggggcctgcctgcaggccccccgatagctcctacaacagcctgtactccacctgcctggagctgttcaagttcaccatcggcatgggcgacctggagttcactgagaactatgacttcaaggctgtcttcatcatcctgctgctggcctatgtaattctcacctacatcctcctgctcaacatgctcatcgccctcatgggtgagactgtcaacaagatcgcacaggagagcaagaacatctggaagctgcagagagccatcaccatcctggacacggagaagagcttccttaagtgcatgaggaaggccttccgctcaggcaagctgctgcaggtggggtacacacctgatggcaaggacgactaccggtggtgcttcagggtggacgaggtgaactggaccacctggaacaccaacgtgggcatcatcaacgaagacccgggcaactgtgagggcgtcaagcgcaccctgagcttctccctgcggtcaagcagagtttcaggcagacactggaagaactttgccctggtcccccttttaagagaggcaagtgctcgagataggcagtctgctcagcccgaggaagtttatctgcgacagttttcagggtctctgaagccagaggacgctgaggtcttcaagagtcctgccgcttccggggagaagtga

SEQ ID NO: 8 - protein, 839 aa, Homo Sapiens [sequence omitted]

(19) United States
(12) Patent Application Publication
(10) Pub. No.: US 2005/0266435 A1
(43) Pub. Date: Dec. 1, 2005

(54) VR1 receptors and uses thereof

(76) Inventors: David Hackos, San Francisco, CA (US); Tito A. Serafini, San Mateo, CA (US); Nina Orike, San Francisco, CA (US)

Correspondence Address: KLAUBER & JACKSON, 411 HACKENSACK AVENUE, HACKENSACK, NJ, 07601, US

(21) Appl. No.: 11/076,431

(22) Filed: Mar. 9, 2005

Related U.S. Application Data
(60) Provisional application No. 60/551,570, filed on Mar. 9, 2004.

(51) Int. Cl. C12Q 1/68; G01N 33/53; G01N 33/567

(52) U.S. Cl. 435/6; 435/7.2

(57) ABSTRACT

The present invention relates to nucleic and amino acid sequences encoding vanilloid receptors which are vanilloid-insensitive. The present invention provides mutant vanilloid receptors which are insensitive to vanilloid, but which are capable of responding to low pH, heat and other receptor modulators. The invention particularly provides mutant vanilloid insensitive human VR1 receptors. The invention also relates to methods and assays for screening for vanilloid receptor modulators that act independent of the vanilloid binding site and modulate receptor signals independent of a functional vanilloid, or capsaicin, response. The invention further provides methods of modulating the vanilloid receptor, independent of vanilloid response.

### CROSS-REFERENCE TO RELATED APPLICATIONS

The present application claims priority from co-pending U.S. Provisional Application Ser. No. 60/551,570, filed Mar. 9, 2004, pursuant to 35 U.S.C. § 119, which is incorporated herein by reference in its entirety.

### FIELD OF THE INVENTION

The present invention relates to nucleic and amino acid sequences encoding vanilloid receptors which are insensitive to vanilloids, do not bind vanilloids and/or do not signal in response to vanilloids. The invention also relates to methods and assays for screening for vanilloid receptor modulators that act independent of a functional vanilloid binding site and/or modulate receptor signals independent of a functional vanilloid, or capsaicin, response.

### BACKGROUND OF THE INVENTION

The sensation of pain can be triggered by any number of physical or chemical stimuli. In mammals, the peripheral terminals of a group of specialized small diameter sensory neurons, termed “nociceptors” mediate this response to a potentially harmful stimulus, including noxious chemical, mechanical, or thermal stimuli. These neurons, whose cell bodies are located in various sensory ganglia, transmit information regarding tissue damage to pain processing centers in the spinal cord and brain. Nociceptors are characterized, in part, by their sensitivity to capsaicin, a vanilloid and a natural product of capsicum peppers that is the active ingredient of many “hot” and spicy foods.

The effects of vanilloids are mediated via a specific nerve membrane receptor, the vanilloid receptor. There are specific physico-chemical requirements for receptor activation and a selective antagonist, capsazepine, is available. The receptor is coupled to a non-specific membrane cation channel that is preferentially permeable to calcium and sodium ions. This receptor is also activated by increases in temperature in the noxious range, suggesting that it functions as a transducer of painful thermal stimuli in vivo.

The best known vanilloid is capsaicin, the main pungent ingredient in hot chili peppers. Capsaicin elicits a sensation of burning pain by selectively activating sensory neurons that convey information about noxious stimuli to the central nervous system. Activation is followed by loss of further sensitivity to capsaicin, insensitivity to noxious heat and chemical stimuli and loss of the ability to release sensory neuro-chemicals involved in neurotransmission and in inflammation. These reversible effects have been exploited to produce novel analgesic and anti-inflammatory substances and used to study the physiological function of the sensory nervous system. With high doses and prolonged exposure, capsaicin can cause selective neurotoxicity, especially when given experimentally to neonatal animals. With prolonged exposure, these terminals become insensitive to capsaicin, as well as to other noxious stimuli (Szolcsanyi in Capsaicin in the Study of Pain (ed. Wood) pgs. 255-272 (Academic Press, London, 1993)). This latter phenomenon of nociceptor desensitization underlies the seemingly paradoxical use of capsaicin as an analgesic agent in the treatment of painful disorders ranging from viral and diabetic neuropathies to rheumatoid arthritis (Campbell in Capsaicin and the Study of Pain (ed. Wood) pgs. 255-272 (Academic Press, London, 1993); Szallasi et al. (1996) Pain 68:195-208). While some of this decreased sensitivity to noxious stimuli may reflect reversible changes in the nociceptor, such as depletion of inflammatory mediators, the long-term loss of responsiveness can be explained by death of the nociceptor or destruction of its peripheral terminals following capsaicin exposure (Jancso et al. (1977) Nature 270:741-743; Szolcsanyi, supra).

The vanilloid receptor channel is not affected by conventional ion channel blockers, but can be blocked by ruthenium red and synthetic arginine-rich hexapeptides. The arginine-containing natural endogenous peptide, dynorphin, is also able to prevent channel opening. Another naturally occurring phorbol that binds to the vanilloid receptor, resiniferatoxin (RTX), is a highly potent irritant isolated from Euphorbia resinefera, and [3H]-RTX has been used to characterize the vanilloid receptor in sensory tissues and to map its distribution in the body. RTX has a non-pungent analogue, Phorbol 12-phenylacetate 13 acetate 20-homovanillate (PPAHV). These studies confirm that vanilloid receptors are highly expressed in sensory neurons, but are also present (though at much lower levels) in the brain, as well as in non-neuronal tissues such as the kidney, the lung, and the spleen. More recently, Pan et al have reported that capsaicin receptors are expressed in the heart and may be responsible for triggering heart attack chest pain (Pan H L et al (2003) J Physiol 551(Pt2):515-523). Receptors occur in the human nervous system, but there are species differences in their distribution. Studies with RTX and PPAHV have suggested that their interaction with the receptor may differ significantly from that of capsaicin (Walpole et al (1996); Liu et al (1998); Szallasi et al (1999); Jerman et al (2000).

The human and rodent vanilloid receptors, VR1, have been cloned and shown to consist of 838 amino acids with a molecular weight of 95 kDa (Caterina et al (1997) Nature 389:816-824; Tominiga et al (1998) Neuron 21:531-543; U.S. Pat. No. 6,335,180; Hayes et al (2000) Pain 88:207-217; McIntyre et al (2001) Br J Pharmacol 132:1084-1094). The VR1 receptor is activated by capsaicin, low pH solutions and temperatures greater than 42° C. and is a non-selective cation channel with high permeability for divalent cations. It contains a heat-sensitive subunit that can explain the burning sensation induced by capsaicin activation. Transgenic animal studies have confirmed that mice lacking the VR1 gene demonstrate normal responses to mechanical stimuli, but reduced thermal hyperalgesia in models of inflammatory pain and lack vanilloid-evoked pain behavior (Caterina et al (2000) Science 288:306-313; Davis et al (2000) Nature 405:183-187). A second VR1 homologue termed the Vanilloid-Receptor-like porotein-1 (VRL-1) does not respond to capsaicin or protons but is sensitive to heat at temperatures above about 52° C. (Caterina et al (1999) Nature 398:436-441; U.S. Pat. No. 6,335,180).

The identity of a single endogenous ligand for the vanilloid receptor is as yet unresolved. Noxious heat and concentrations of protons that occur during tissue acidosis and inflammation have been proposed to be endogenous activators or dynamic modulators of the vanilloid receptor. Rat VR1 is activated by endogenous lipoxygenase products that are likely to be expressed in inflamed tissues (Hwang et al (2000) PNAS 97:6155-6160). The endogenous cannabinoid, anandamide, an activator of the cannabinoid (CB) receptor, has also been shown to activate VR1 (Zygmunt et al (1999) Nature 400:452-457; Smart et al (2000) Br J Pharmacol 129:227-230). Other inflammatory agents such as PGE₂ which do not directly activate VR1, sensitize the vanilloid receptor to other noxious stimuli (Kress et al (1997) Neurosci Lett 224:1-4; Vyklicky et al (1998) J Neurophsiol 79:670-676).

Capsaicin and other vanilloids have been used beneficially as topical applications in many pain conditions that do not respond to conventional analgesics. These include nerve injury pain, such as post-herpetic neuralgia or post-mastectomy pain, as well as intractable migraine and chronic inflammatory pain, such as rheumatoid arthritis. Desensitization to vanilloids is a promising therapeutic approach to mitigate neuropathic pain and pathological conditions (e.g. vasomotor rhinitis) in which neuropeptides released from primary sensory neurons play a major role.

In addition, modulation of vanilloid receptors has possible therapeutic utility in disorders of urinary bladder function, in chronic itch disorders and in controlling emesis. The potential for broad therapeutic application has stimulated development of vanilloids, such as olvanil and nuvanil, with reduced irritant side effects as well as exploration of resiniferatoxin and non-vanilloid substances as a therapeutic chemical prototypes. The cloning of the VR1 receptor has accelerated the search for selective ligands with which to characterize the receptor while also stimulating interest in drug discovery in this rapidly moving field.

The use of capsaicin, however, is severely limited by its irritancy, and the synthesis of novel vanilloids with an improved pungency/desensitization ratio is an on-going objective. These vanilloid drugs would prevent capsaicin from interacting with the receptor and therefore block capsaicin-induced activation of the channel. However, blocking the channel in this way does not necessarily prevent endogenous activators (anandamide, acid, heat, etc.) from activating VR1. Therefore, there is a need in the art for the discovery of drugs that modulate the vanilloid receptor by interacting with the receptor independent of a functional capsaicin binding site that may be more physiologically relevant. There is a need for methods to screen for vanilloid receptor, including VR1, modulators, agonists and antagonists, that specifically interact with vanilloid insensitive receptors, interacting with the vanilloid receptor independent of a functional capsaicin-binding site

The citation of references herein shall not be construed as an admission that such is prior art to the present invention.

### SUMMARY OF THE INVENTION

The invention provides methods for identifying and screening compounds that modulate vanilloid-insensitive vanilloid receptors. The present invention provides methods for identifying compounds that modulate vanilloid receptor polypeptide, wherein said compounds bind or otherwise interact with the vanilloid receptor, particularly the VR1 receptor, independent of a functional capsaicin-binding site or a functioning capsaicin response. In particular, the invention provides methods for identifying compounds that modulate human vanilloid receptor VR1 polypeptide, wherein said compounds bind or otherwise interact with the human VR1 independent of a functional vanilloid-binding site or a functioning vanilloid response. The invention provides methods for identifying compounds that modulate vanilloid receptor polypeptide utilizing vanilloid-insensitive receptors. Thus, the invention relates to screening for agonists or antagonists of the vanilloid receptors that are not vanilloids and/or which do not interact with the vanilloid receptor through the vanilloid binding site or act independent of functional vanilloid binding or vanilloid-responsiveness.

The invention provides methods for detecting a compound that modulates the vanilloid receptor, comprising:
(a) contacting a sample suspected of containing a vanilloid receptor modulating compound with a host cell expressing a vanilloid receptor which is insensitive to vanilloid; and
(b) detecting an alteration of a cellular response associated with vanilloid receptor activity in the vanilloid receptor expressing cost cell.

The invention provides methods for detecting a compound that modulates the VR1 receptor, comprising:
(a) contacting a sample suspected of containing a VR1 receptor modulating compound with a host cell expressing a VR1 receptor which is insensitive to vanilloid; and
(b) detecting an alteration of a cellular response associated with VR1 receptor activity in the VR1 receptor expressing cost cell.

The present invention relates to a method for identifying compounds that bind a mutant capsaicin receptor polypeptide, particularly compounds that bind a vanilloid receptor polypeptide independent of the vanilloid binding site, or a functioning vanilloid-binding site and affect a cellular response associated with vanilloid receptor biological activity, including intracellular calcium flux.

In one aspect, the invention provides a method for detecting a compound that modulates the vanilloid receptor, wherein said compound binds or otherwise interacts with the vanilloid receptor independent of the vanilloid-binding site, comprising:
(a) contacting a sample suspected of containing a vanilloid receptor modulating agent/compound with a host cell expressing a mutant vanilloid receptor which does not bind vanilloid compounds; and
b) detecting an alteration of a cellular response associated with vanilloid receptor activity in the mutant vanilloid receptor expressing host cell.

In one aspect, the invention provides a method for detecting a compound that modulates the VR1 receptor, wherein said compound binds or otherwise interacts with the VR1 receptor independent of the vanilloid-binding site, comprising:
(a) contacting a sample suspected of containing a VR 1 receptor modulating agent/compound with a host cell expressing a mutant VR1 receptor which does not bind vanilloid compounds; and
(b) detecting an alteration of a cellular response associated with VR1 receptor activity in the mutant VR1 receptor expressing host cell.

In a further aspect, the invention provides a method for detecting a compound that modulates the VR1 receptor, wherein said compound binds or otherwise interacts with the VR1 receptor independent of the vanilloid-binding site, comprising:
(a) contacting a sample suspected of containing a VR1 receptor modulating agent/compound with a host cell expressing a mutant VR1 receptor which does not bind vanilloid compounds;
(b) detecting an increase in intracellular calcium concentration in the mutant VR1 receptor expressing host cell.

The invention relates to a method for screening for biologically active agents that modulate VR1 receptor function independent of vanilloid binding or response comprising:
(a) combining a candidate agent with any one of:
   (i) a mammalian VR1 receptor polypeptide which does not bind and/or respond to vanilloid compounds;
   (ii) a mammalian VR1 receptor-like polypeptide which does not bind and/or respond to vanilloid compounds;
   (iii) a cell containing a nucleic acid encoding a mammalian VR1 receptor polypeptide which does not bind and/or respond to vanilloid compounds; and
(b) determining the effect of said agent on VR1 receptor function.

The present invention features mutant vanilloid receptor polypeptides, specifically mutant capsaicin receptor polypeptides, as well as nucleotide sequences encoding mutant vanilloid receptor polypeptides, which do not bind and/or respond to vanilloid compounds. In related aspects the invention features expression vectors and host cells comprising polynucleotides that encode mutant vanilloid receptor/mutant capsaicin receptor polypeptide wherein the mutant receptor does not bind and/or does not respond to vanilloid compounds. In other related aspects, the invention features transgenic animals expressing mutant capsaicin receptor, due to the presence of mutant capsaicin receptor-encoding polynucleotide sequence. The present invention also relates to antibodies that bind specifically to a mutant capsaicin receptor polypeptide, wherein the mutant does not bind and/or does not respond to vanilloid compounds.

The invention provides an isolated mutant human vanilloid receptor which is insensitive to vanilloid. The invention provides an isolated human VR1 receptor polypeptide wherein the polypeptide does not bind to and/or respond to vanilloid compounds.

In one aspect, the invention provides an isolated human VR1 receptor polypeptide wherein the polypeptide does not bind to and/or respond to capsaicin wherein one or more amino acids in the vanilloid binding site are altered such that the VR1 receptor does not bind to and/or respond to capsaicin.

In a particular aspect, a VR1 receptor polypeptide is provided wherein one or more of amino acids selected from the group of amino acids 505, 506, 507, 508, 509, 510, 511, 512, 513, 514, 515, 516 and 517 is replaced with another amino acid. In particular, one or more of amino acids 505, 506, 507, 508, 509, 510, 511, 512, 513, 514, 515, 516 and 517 is replaced with a non-conservative amino acid.

The invention further provides an isolated VR1 receptor polypeptide comprising the amino acid sequence as set out in FIG. 8.

The invention includes a nucleic acid sequence which encodes a vanilloid insensitive VR1 receptor, or a fragment thereof having receptor activity, selected from the group consisting of:
(a) the DNA sequence of FIG. 1 (SEQ ID NO:);
(b) the DNA sequence of FIG. 2 (SEQ ID NO:);
(c) the DNA sequence of FIG. 3 (SEQ ID NO:);
(d) DNA sequences that hybridize to any of the foregoing DNA sequences under standard hybridization conditions; and
(e) DNA sequences that code on expression for an amino acid sequence encoded by any of the foregoing DNA sequences.

The invention thus provides a nucleic acid sequence encoding a mutant human vanilloid receptor polypeptide wherein one or more of amino acids 505, 506, 507, 508, 509, 510, 511, 512, 513, 514, 515, 516 and 517 is replaced with another amino acid.

The invention further provides a nucleic acid sequence encoding the vanilloid receptor polypeptide of FIG. 8. In a particular embodiment, the invention provides a nucleic acid sequence comprising the sequence as set out in FIG. 8. In a further embodiment, the nucleic acid has the sequence as set out in FIG. 8.

The invention provides a nucleic acid vector for recombinant expression of a human VR1 receptor which does not bind and/or respond to vanilloid comprising a nucleic acid sequence encoding a human VR1 receptor which does not bind and/or respond to vanilloid, operatively linked to an expression control sequence. The invention includes host cells that have been stably transfected with a nucleic acid vector for recombinant expression of a human VR1 receptor which does not bind and/or respond to vanilloid.

In one aspect, the invention provides an antibody specific to a vanilloid receptor the receptor to which said antibody is raised having the following characteristics:
(a) insensitive to vanilloid;
(b) sensitive to low pH; and
(c) sensitive to heat.

In one aspect, the invention provides an antibody specific to a VR1 receptor the receptor to which said antibody is raised having the following characteristics:
(a) insensitive to vanilloid;
(b) sensitive to low pH; and
(c) sensitive to heat.

The vanilloid-insensitive receptor specific antibody may be a polyclonal antibody or a monoclonal antibody.

The invention includes a method of testing the ability of a drug or other entity to modulate the activity of a vanilloid insensitive receptor which comprises
(a) culturing a colony of test cells which has a vanilloid insensitive receptor in a growth medium containing a known vanilloid receptor agonist or antagonist or in the presence of heat or low pH;
(b) adding the drug under test; and
(c) measuring the reactivity of said known agonist or antagonist with the vanilloid insensitive receptor on said colony of test cells,
   wherein said vanilloid insensitive receptor has the following characteristics:
(i) does not bind or respond to capsaicin
(ii) does respond to low pH;
(iii) does respond to heat.

In a particular embodiment of this method the vanilloid insensitive receptor polypeptide has one or more of amino acids selected from the group of amino acids 505, 506, 507, 508, 509, 510, 511, 512, 513, 514, 515, 516 and 517 replaced with another amino acid.

Yet another aspect of the invention relates to use of mutant vanilloid receptor polypeptides which do not bind and/or do not respond to vanilloid and specific antibodies thereto for the diagnosis and treatment of human disease and painful syndromes. Such diseases and painful syndromes include, but are not limited to pain, chronic pain, neuropathic pain, postoperative pain, rheumatoid arthritic pain, osteoarthritic pain, back pain, visceral pain, cancer pain, algesia, neuralgia, migraine, neuropathies, diabetic neuropathy, sciatica, HIV-related neuropathy, post-herpetic neuralgia, fibromyalgia, nerve injury, ischemia, neurodegeneration and neurodegenerative disorders, stroke, post stroke pain, multiple sclerosis, inflammatory disorders, irritable bowel syndrome, inflammatory bowel disease and burns.

The invention further relates to biologically active agents that modulate vanilloid receptor function, including VR1 receptor and vanilloid receptor-like receptor function, independent of vanilloid binding or response, particularly identified or characterized in the methods of screening provided herein, and their use in the diagnosis and treatment of human disease and painful syndromes. Such diseases and painful syndromes include, but are not limited to pain, chronic pain, neuropathic pain, postoperative pain, rheumatoid arthritic pain, osteoarthritic pain, back pain, visceral pain, cancer pain, algesia, neuralgia, migraine, neuropathies, diabetic neuropathy, sciatica, HIV-related neuropathy, post-herpetic neuralgia, fibromyalgia, nerve injury, ischemia, neurodegeneration and neurodegenerative disorders, stroke, post stroke pain, multiple sclerosis, inflammatory disorders, irritable bowel syndrome, inflammatory bowel disease and burns.

In another aspect the invention features transgenic, non-human mammals expressing a mutant vanilloid receptor-encoding transgene, which does not bind and/or does not respond to vanilloid, and use of such transgenic mammals in screening of candidate vanilloid receptor agonist and antagonist compounds.

Other objects and advantages will become apparent to those skilled in the art from a review of the following description which proceeds with reference to the following illustrative drawings.

### BRIEF DESCRIPTION OF THE DRAWINGS

FIG. 1 depicts the nucleic and amino acid sequence of the Y511A VR1 mutant receptor.

FIG. 2 depicts the nucleic and amino acid sequence of the Y511C VR1 mutant receptor.

FIG. 3 depicts the nucleic and amino acid sequence of the S512Y VR1 mutant receptor.

FIG. 4 shows typical results obtained from the mutant VR1 receptor Y511C using calcium imaging with fura-2. Experiments were done using a normal saline (pH 7.6) wash solution as the control solution. Under these conditions, a low pH saline solution (pH 5.1) causes a large influx of calcium into the transfected 293 cells as a result of VR1 activation. These mutant receptors have no detectable response to 250 nM capsaicin and are thus capsaicin-insensitive channels. 100 nM Compound A (a putative VR1 receptor antagonist) is shown to completely block the effect of low pH on these channels. Thus, this drug can block VR1 even when the capsaicin binding site has been completely disrupted. We have also verified that the mutant channels Y511A and S512Y similarly make acid-sensitive and capsaicin-insensitive channels that can also be at least partially blocked by Compound A.

FIG. 5 shows results from a screen using high-throughput ratiometric fluorimetry using a Molecular Devices Flexstation, depicting plots of the fluorescence ratio versus time. The mutant Y511C VR1 receptor is expressed stably in HEK 293 cells, loaded with fura-2, and analyzed by measuring the ratio of fluorescence excited by 340 nm and 380 nm. The control plots demonstrate the effects of recording the cells either with (positive control) or without (negative control) a low pH (pH 5.1) stimulus to open the channels. The compound plots, Compound A through Compound G, show the effect of a 300 nM concentration of each compound added 20 sec. before the addition of the low pH stimulus. Compound A significantly blocks the mutant channel, while the other compounds tested in this screen show varying amounts of block.

FIG. 6 depicts specific binding of 3H-Resiniferatoxin (RTX) to membranes of 293 HEK cells expressing the human VR1 and mutant VR1 receptor Y511C. Binding of 3H-RTX (0.5 nM) to the membranes was conducted in the absence (labeled total binding) or presence (labeled non specific binding) of unlabeled RTX (200 nM) as indicated. This indicates that RTX shows no significant binding to the mutant VR1 receptor in this concentration range.

FIG. 7 depicts a comparison of the nucleic acid sequences of mutant Y511A, Y511C, and S512Y VR1 receptors.

FIG. 8 depicts the nucleic and amino acid sequence of the unmutated human VR1 receptor, a vanilloid-responsive receptor, which was the starting sequence for the mutants generated and studies described herein.

FIG. 9A-9D depicts results demonstrating that capsaicin fails to activate the mutant VR1 receptors Y511A and Y511C expressed in Xenopus oocytes. A) A typical recording of the wild-type human VR1 receptor showing the large response elicited by the application of 10 μM capsaicin. The mutant VR1 receptors Y511C (B) and Y511A (C) have no detectable capsaicin response even at capsaicin concentrations as high as 100 μM. The mutant receptor S512Y (D) shows a small capsaicin response at 100 μM capsaicin but no detectable response at 10M capsaicin (data not shown).

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

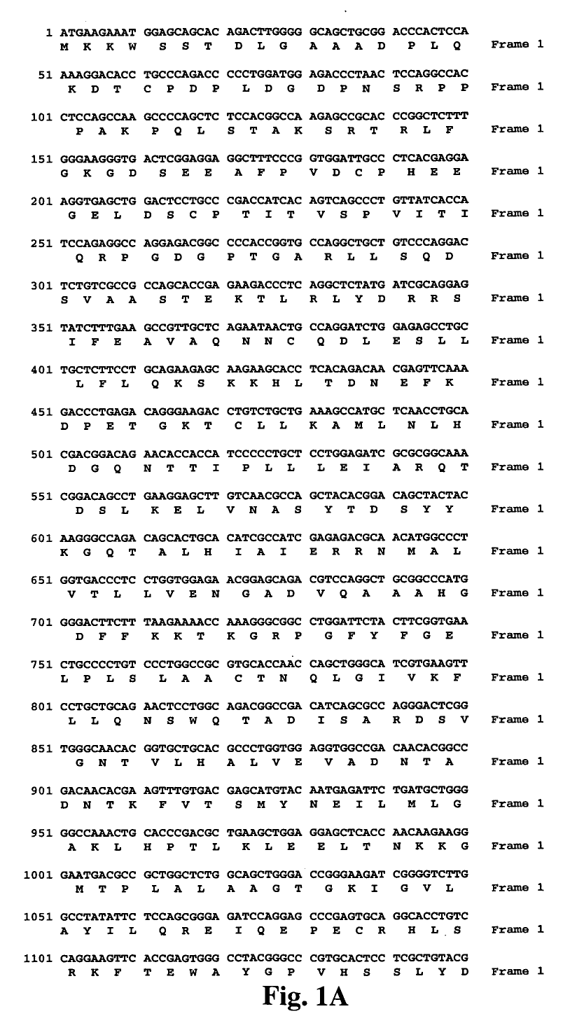

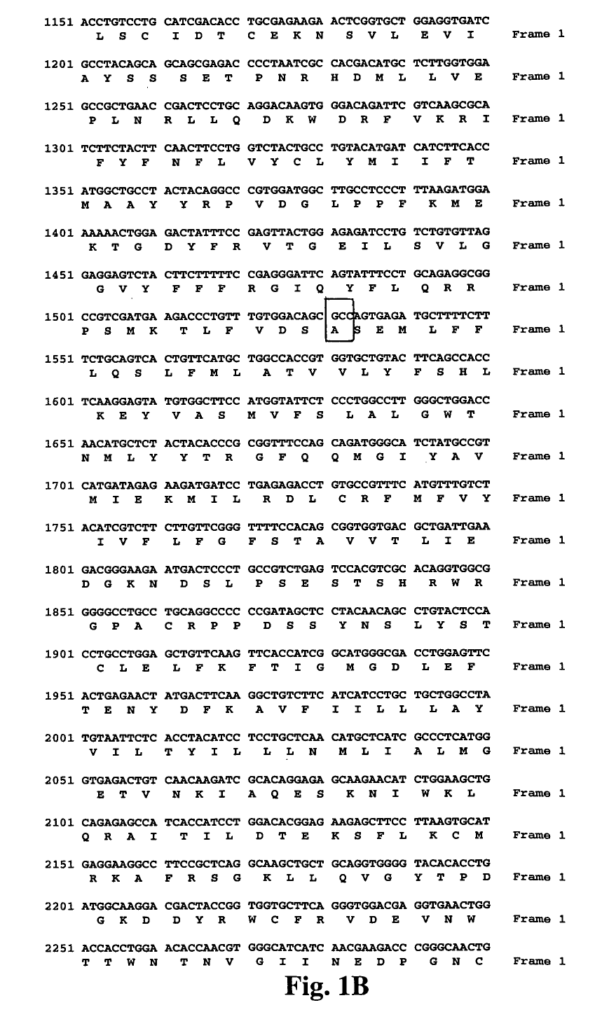

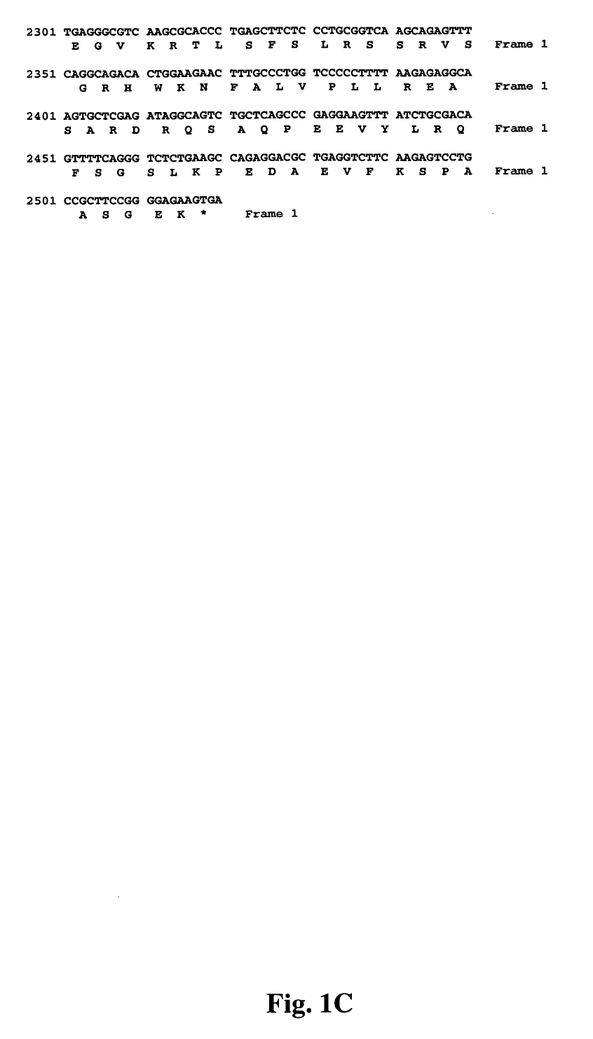

The terms “vanilloid receptor”, “VR”, “VR1 receptor”, “VR1” and any variants not specifically listed, may be used herein interchangeably, and as used throughout the present application and claims refer to proteinaceous material including single or multiple proteins, particularly including a single protein or complex of one or more protein (including homomers and heteromers) and extends to those proteins having the amino acid sequence data described herein and presented in FIGS. 1, 2, 3 and 6, and the profile of activities set forth herein and in the Claims. In particular these vanilloid receptors include vanilloid receptors which are insensitive to vanilloid, by virtue of not binding or otherwise not responding to vanilloid compounds, including for instance capsaicin. Accordingly, proteins displaying substantially equivalent or altered activity, wherein they are insensitive to vanilloid, particularly capsaicin, but are sensitive to other agonists or antagonists, including for instance one or more of low pH and heat, are likewise contemplated. These modifications may be deliberate, for example, such as modifications obtained through site-directed mutagenesis, or may be accidental, such as those obtained through mutations in hosts that are producers of the complex or its named subunits. Also, the terms “vanilloid receptor”, “VR”, “VR1 receptor”, “VR1” are intended to include within their scope proteins specifically recited herein as well as all substantially homologous analogs and allelic variations. Vanilloid receptor-like polypeptides are further included within the scope of the vanilloid receptor herein, including single proteins or complexes of more than one protein, or other components, including homomers and heteromers, whose function ordinarily is or can be modulated by a vanilloid compound.

Vanilloid receptor-like sequences included and contemplated herein include those which are known and which are yet unknown, and which are characterized in having amino acid sequences or structural or functional characteristics which mimic or are similar to VR1. Exemplary but not limiting vanilloid receptor-like polypeptides include, but are not limited to, those described by Cortright and Krause (VRL-1) (US Patent Publication No. 2003/0087389), by Shinjo and Yabuuchi (VRL-2) (US Patent Publication No. 2003/0017527), by Davis and Smith (VRL-3) (PCT International Publication Number WO 03/074562) and by Partiseti and Renard (PCT International Publication Number WO 99/46377).

“Vanilloid-insensitive” receptors as used herein refer to and include receptors which are insensitive to one or more vanilloid(s), fail to demonstrate normal or significant receptor channel response or activation (including for instance the influx of calcium or channel opening) on incubation with or exposure to vanilloid(s), and/or fail to bind or significantly associate with vanilloid compound(s). Failure to respond or bind includes the complete absence of response or binding or the existence of low or significantly reduced binding or response, particularly on comparative measurement with a vanilloid responsive or normal vanilloid receptor. Vanilloid compounds particularly refer to known and recognized vanilloid(s), including for instance capsaicin, and include compounds which may not have a chemical structural component that is strictly classified as vanilloid, but that nonetheless possess a related group such as a vanillyl chemical structural component. Binding may be determined by methods known and recognized in the art, including as described herein, including a determination of a physical association or interaction with a vanilloid compound, for instance by assessment of binding of labeled compound such as of ³H RTX. Response or activation may be determined by methods known and recognized in the art, including as described herein, including by measurement of channel opening in oocytes and/or calcium influx as measured by indicator dye(s). Vanilloid insensitive receptors include homomer and heteromer receptors. Vanilloid insensitive receptors contemplated herein include the particular exemplary mutant receptors provided herein as well as alternative mutants and natural or allelic variants of vanilloid receptors or vanilloid-like receptors which fail to respond to vanilloid(s). Vanilloid insensitivity may arise by alteration of amino acids involved in receptor interaction with vanilloid(s), for instance in a vanilloid binding site, and/or by alteration of amino acids in other regions of the receptor outside but near the binding site such that the structure or folding of the binding site is altered, and/or by alteration of amino acids in other regions of the receptor such that receptor structure is altered. Vanilloid insensitive receptors particularly include receptors wherein the interaction with and/or receptor response to vanilloid(s) is altered, but wherein the receptors are responsive to other receptor activators including for instance one or more of heat, altered pH, and non-vanilloid activators or antagonists.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryqtic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the methods For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

It should be appreciated that also within the scope of the present invention are DNA sequences encoding vanilloid receptors, particularly vanilloid-insensitive vanilloid receptors, which code for a vanilloid receptor having the same amino acid sequence as any of those depicted in FIGS. 1, 2, 3 or 6 or as described herein, including in Example 1, but which are degenerate to these sequences. By “degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC |
| | or CUA or CUG |
| Isoleucine (Ile or I) | AUU or AUC or AUA |
| Methionine (Met or M) AUG | |
| Valine (Val or V) | GUU or GUC of GUA or GUG |
| Serine (Ser or S) | UCU or UCC or UCA or UCG |
| | or AGU or AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (GIu or E) | GAA or GAG |
| Cysteine (Cys or C) | UGU or UGC |
| Arginine (Arg or R) | CGU or CGC or CGA or CGG |
| | or AGA or AGG |
| Glycine (Gly or G) | GGU or GGC or GGA or GGG |
| Tryptophan (Trp or W) | UGG |
| Termination codon | UAA (ochre) or UAG |
| | (amber) or UGA (opal) |

It should be understood that the codons specified above are for RNA sequences. The corresponding codons for DNA have a T substituted for U.

Mutations can be made in the vanilloid receptor, including in those sequences described herein, such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

The following is one example of various groupings of amino acids:

Amino Acids with Nonpolar R Groups

Alanine, Valine, Leucine, Isoleucine, Proline, Phenylalanine, Tryptophan, Methionine

Amino Acids with Uncharged Polar R Groups

Glycine, Serine, Threonine, Cysteine, Tyrosine, Asparagine, Glutamine

Amino Acids with Charged Polar R Groups (Negatively Charged at Ph 6.0)

Aspartic acid, Glutamic acid

Basic Amino Acids (Positively Charged at pH 6.0)

Lysine, Arginine, Histidine (at pH 6.0)

Another grouping may be those amino acids with phenyl groups:

Phenylalanine, Tryptophan, Tyrosine

Another grouping may be according to molecular weight (i.e., size of R groups):

| | |
|---|---|
| Glycine | 75 |
| Alanine | 89 |
| Serine | 105 |
| Proline | 115 |
| Valine | 117 |
| Threonine | 119 |
| Cysteine | 121 |
| Leucine | 131 |
| Isoleucine | 131 |
| Asparagine | 132 |
| Aspartic acid | 133 |
| Glutamine | 146 |
| Lysine | 146 |
| Glutamic acid | 147 |
| Methionine | 149 |
| Histidine (at pH 6.0) | 155 |
| Phenylalanine | 165 |
| Arginine | 174 |
| Tyrosine | 181 |
| Tryptophan | 204 |

Particularly preferred substitutions are:
Lys for Arg and vice versa such that a positive charge may be maintained;
Glu for Asp and vice versa such that a negative charge may be maintained;
Ser for Thr such that a free —OH can be maintained; and
Gln for Asn such that a free NH₂ can be maintained.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces—turns in the protein's structure.

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to modulate, and preferably reduce by at least about 30 percent, more preferably by at least 50 percent, most preferably by at least 90 percent, a clinically significant change in the receptor activity of a target cell or cellular mass, or other feature of pathology such as for example the degree of or amount of pain experienced or the response to a noxious stimulus, as may attend its presence and activity.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20^{N}C below the predicted or determined Tₘ with washes of higher stringency, if desired.

The invention provides methods for identifying and screening compounds that modulate vanilloid-insensitive vanilloid receptors. The present invention provides methods for identifying compounds that modulate vanilloid receptor polypeptide, wherein said compounds bind or otherwise interact with the vanilloid receptor, particularly the VR1 receptor, independent of a functional capsaicin-binding site or a functioning capsaicin response. In particular, the invention provides methods for identifying compounds that modulate human vanilloid receptor VR1 polypeptide, wherein said compounds bind or otherwise interact with the human VR1 receptor independent of a functional vanilloid-binding site or a functioning vanilloid response. The invention provides methods for identifying compounds that modulate vanilloid receptor polypeptide utilizing vanilloid-insensitive receptors. Thus, the invention relates to screening for agonists or antagonists of the vanilloid receptors that are not vanilloids and which do not interact with the vanilloid receptor through the vanilloid binding site or act independent of functional vanilloid binding or vanilloid-responsiveness.

The invention provides methods for detecting a compound that modulates the vanilloid receptor, comprising:
(a) contacting a sample suspected of containing a vanilloid receptor modulating compound with a host cell expressing a vanilloid receptor which is insensitive to vanilloid; and
(b) detecting an alteration of a cellular response associated with vanilloid receptor activity in the vanilloid receptor expressing cost cell.

The invention provides methods for detecting a compound that modulates the VR1 receptor, comprising:
(a) contacting a sample suspected of containing a VR1 receptor modulating compound with a host cell expressing a VR1 receptor which is insensitive to vanilloid; and
(b) detecting an alteration of a cellular response associated with VR1 receptor activity in the VR1 receptor expressing cost cell.

The present invention relates to a method for identifying compounds that bind a mutant capsaicin receptor polypeptide, particularly compounds that bind a vanilloid receptor polypeptide independent of the vanilloid binding site and affect a cellular response associated with vanilloid receptor biological activity, including intracellular calcium flux.

In one aspect, the invention provides a method for detecting a compound that modulates the vanilloid receptor, including VR1 and vanilloid receptor-like receptors, wherein said compound binds or otherwise interacts with the vanilloid receptor independent of the vanilloid-binding site, comprising:
(a) contacting a sample suspected of containing a vanilloid receptor modulating agent/compound with a host cell expressing a mutant vanilloid receptor which does not bind vanilloid compounds; and
(b) detecting an alteration of a cellular response associated with vanilloid receptor activity in the mutant vanilloid receptor expressing host cell.

In one aspect, the invention provides a method for detecting a compound that modulates the VR1 receptor, wherein said compound binds or otherwise interacts with the VR1 receptor independent of the vanilloid-binding site, comprising:
(a) contacting a sample suspected of containing a VR1 receptor modulating agent/compound with a host cell expressing a mutant VR1 receptor which does not bind vanilloid compounds; and
(b) detecting an alteration of a cellular response associated with VR1 receptor activity in the mutant VR1 receptor expressing host cell.

In a further aspect, the invention provides a method for detecting a compound that modulates the VR1 receptor, wherein said compound binds or otherwise interacts with the VR1 receptor independent of the vanilloid-binding site, comprising:
(a) contacting a sample suspected of containing a VR1 receptor modulating agent/compound with a host cell expressing a mutant VR1 receptor which does not bind vanilloid compounds;
(b) detecting an increase in intracellular calcium concentration in the mutant VR1 receptor expressing host cell.

The invention relates to a method for screening for biologically active agents that modulate vanilloid receptor function independent of vanilloid binding or response comprising:
(a) combining a candidate agent with any one of:
   (i) a mammalian VR1 receptor polypeptide which does not bind and/or respond to vanilloid compounds;
   (ii) a mammalian vanilloid receptor-like polypeptide which does not bind and/or respond to vanilloid compounds;
   (iii) a cell containing a nucleic acid encoding a mammalian VR1 receptor polypeptide or vanilloid receptor-like polypeptide which does not bind and/or respond to vanilloid compounds; and
(b) determining the effect of said agent on vanilloid receptor function.

The present invention features mutant vanilloid receptor polypeptides, specifically mutant capsaicin receptor polypeptides, as well as nucleotide sequences encoding mutant vanilloid receptor polypeptides, which do not bind and/or respond to vanilloid compounds. In related aspects the invention features expression vectors and host cells comprising polynucleotides that encode mutant vanilloid receptor/mutant capsaicin receptor polypeptide wherein the mutant receptor does not bind and/or does not respond to vanilloid compounds. In other related aspects, the invention features transgenic animals expressing mutant capsaicin receptor, due to the presence of mutant capsaicin receptor-encoding polynucleotide sequence. The present invention also relates to antibodies that bind specifically to a mutant capsaicin receptor polypeptide, wherein the mutant does not bind and/or does not respond to vanilloid compounds.

The invention provides an isolated mutant human vanilloid receptor which is insensitive to vanilloid. The invention provides an isolated human VR1 receptor polypeptide wherein the polypeptide does not bind to and/or respond to vanilloid compounds.

In one aspect, the invention provides an isolated human VR1 receptor polypeptide wherein the polypeptide does not bind to and/or respond to capsaicin wherein one or more amino acids in the vanilloid binding site are altered such that the VR1 receptor does not bind to and/or respond to capsaicin.

In a particular aspect, a vanilloid receptor polypeptide is provided wherein one or more of amino acids selected from the group of amino acids 505, 506, 507, 508, 509, 510, 511, 512, 513, 514, 515, 516 and 517 is replaced with another amino acid. In a particular aspect, a VR1 receptor polypeptide is provided wherein one or more of amino acids selected from the group of amino acids 505, 506, 507, 508, 509, 510, 511, 512, 513, 514, 515, 516 and 517 is replaced with another amino acid. In particular, one or more of amino acids 505, 506, 507, 508, 509, 510, 511, 512, 513, 514, 515, 516 and 517 is replaced with a non-conservative amino acid.

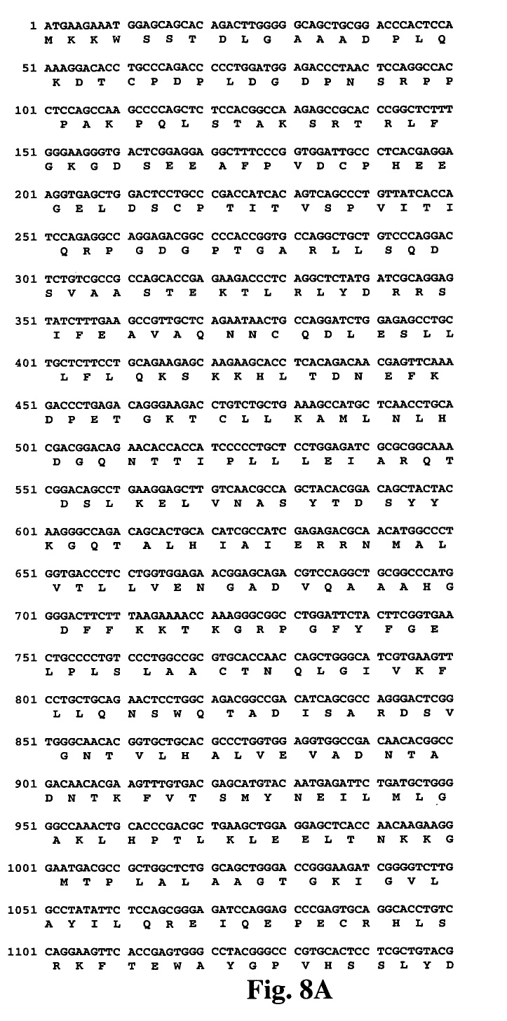

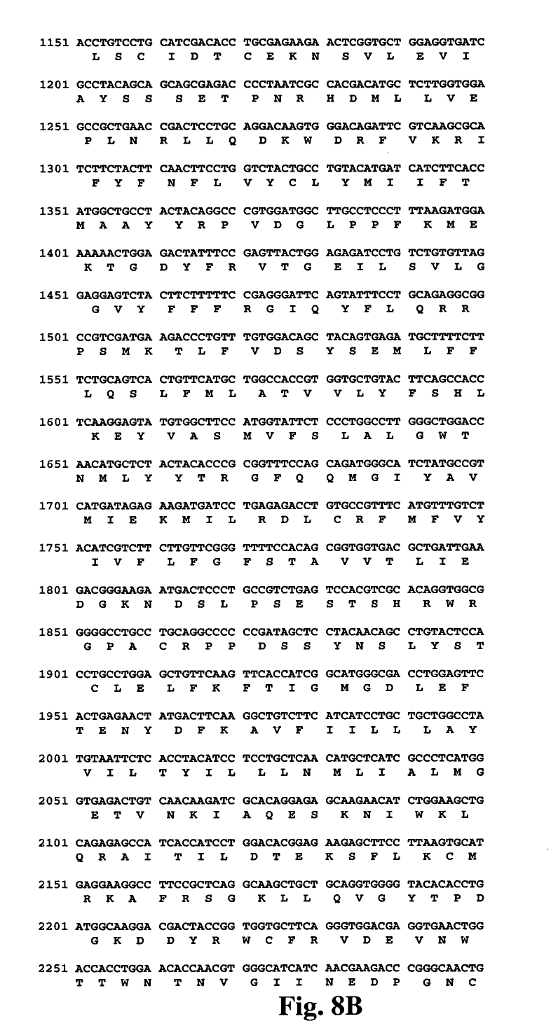

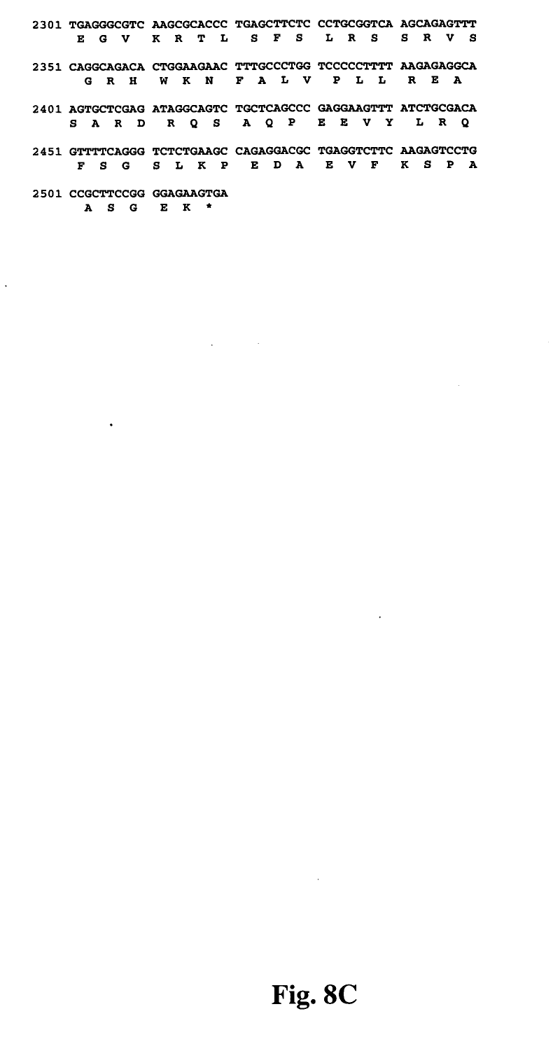

The invention includes a vanilloid receptor polypeptide as set out in FIG. 8, as well as nucleic acid sequences encoding the vanilloid receptor polypeptide of FIG. 8. One nucleic acid embodiment is provided in the nucleic acid sequence depicted in FIG. 8.

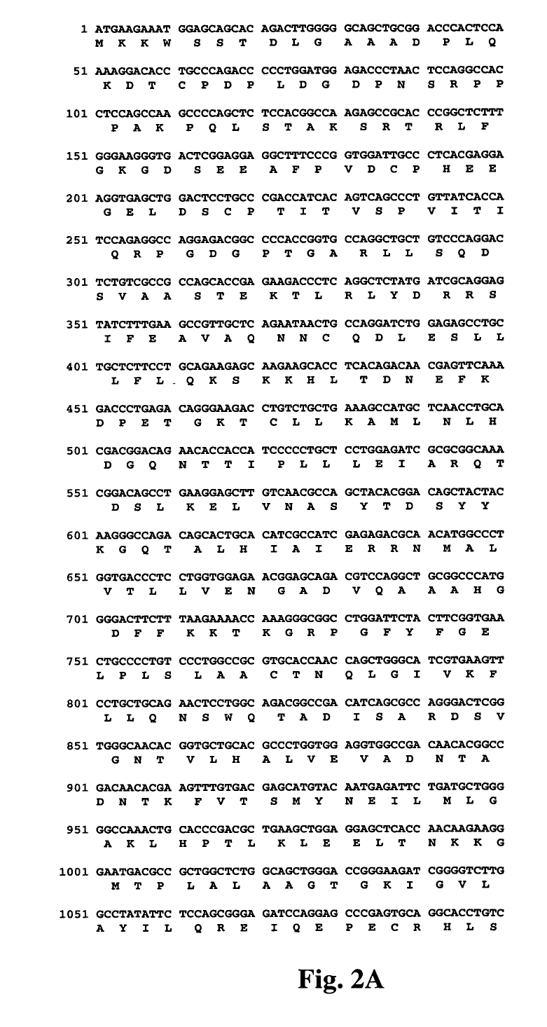

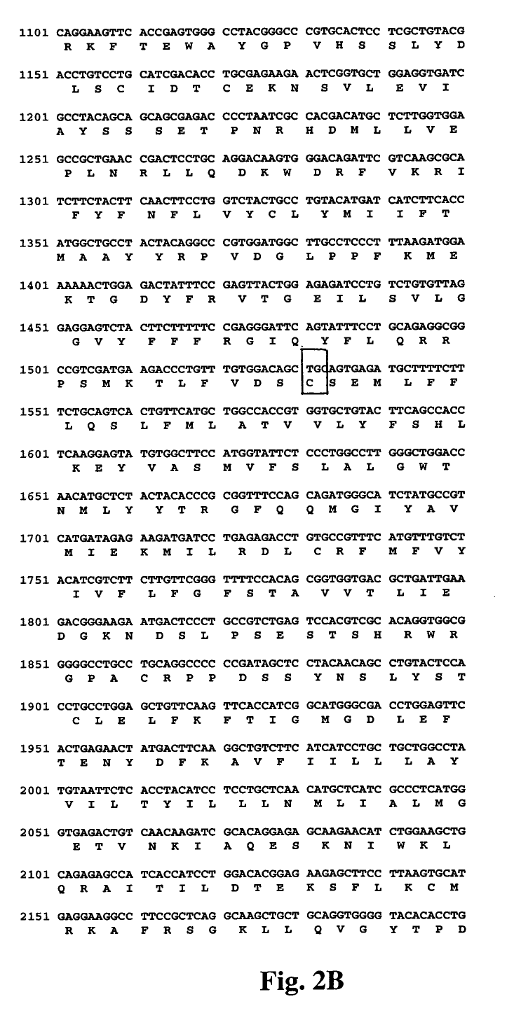

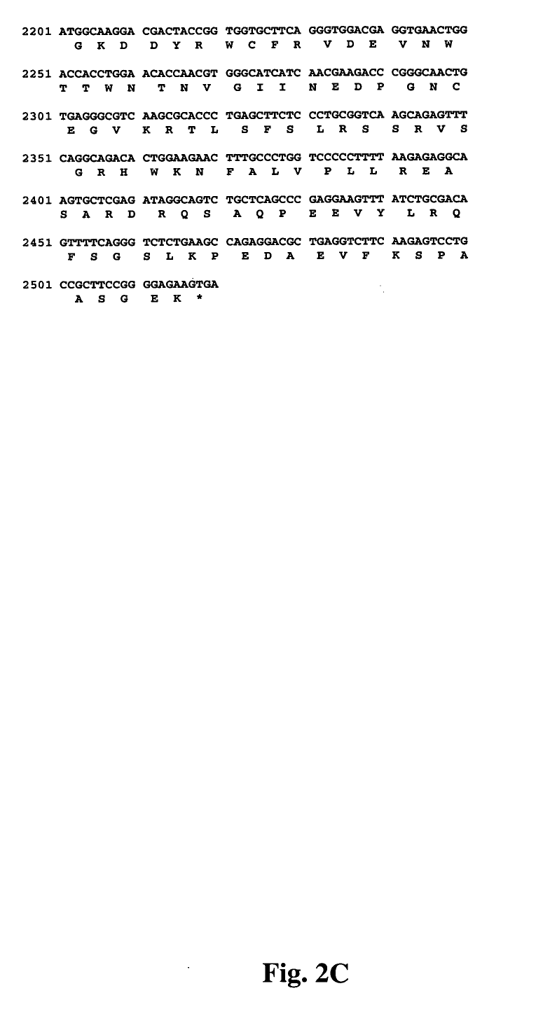

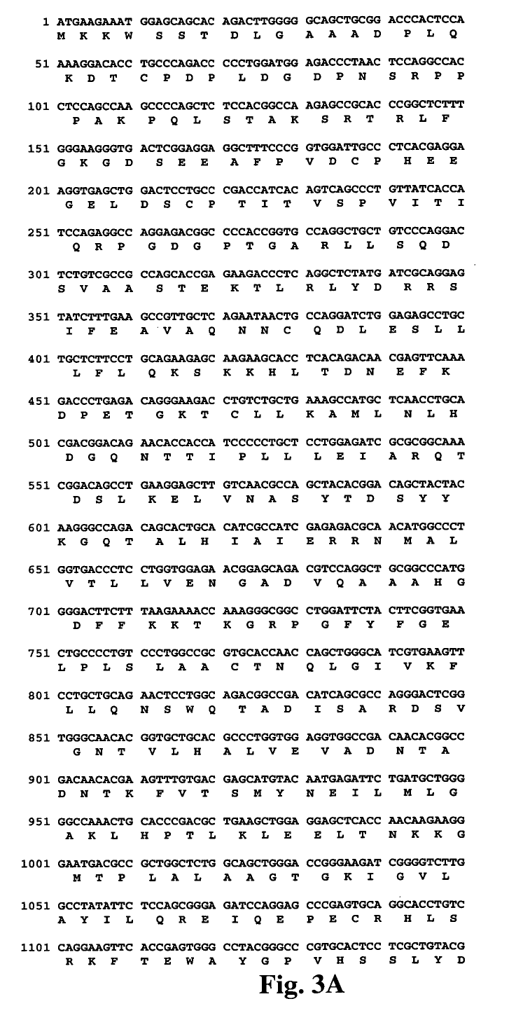

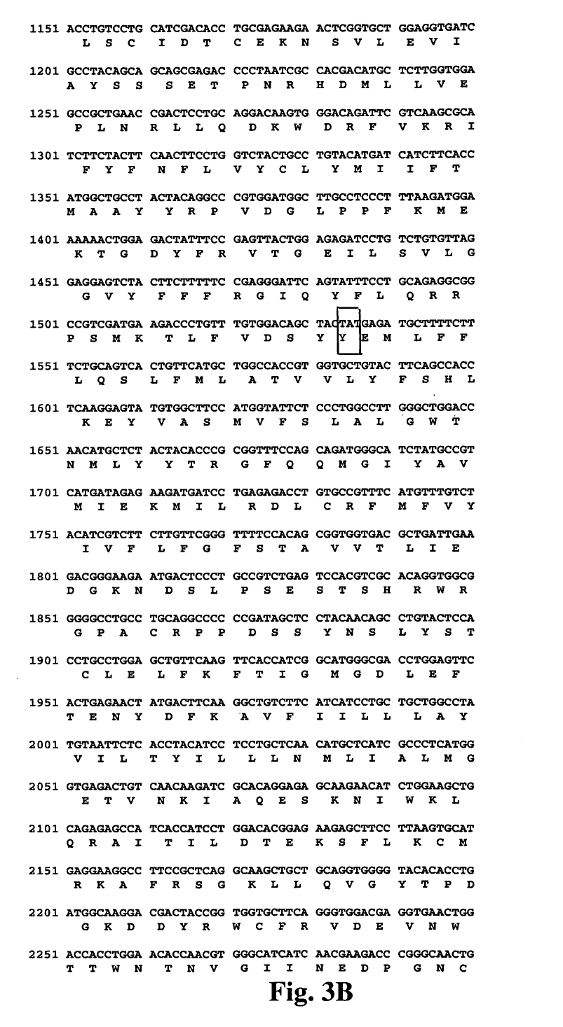

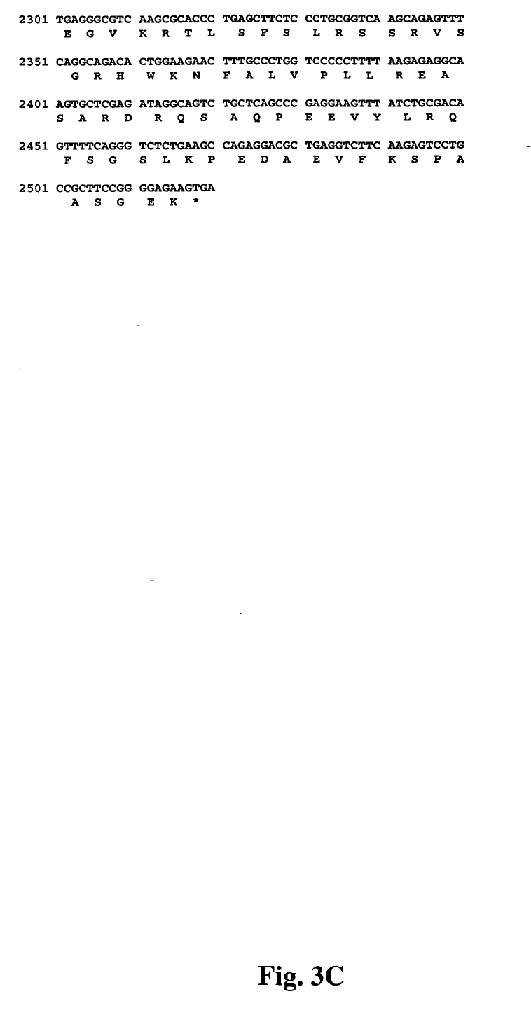

The invention includes a nucleic acid sequence which encodes a vanilloid insensitive VR1 receptor, or a fragment thereof having receptor activity, selected from the group consisting of:
(a) the DNA sequence of FIG. 1 (SEQ ID NO:);
(b) the DNA sequence of FIG. 2 (SEQ ID NO:);
(c) the DNA sequence of FIG. 3 (SEQ ID NO:);
(d) DNA sequences that hybridize to any of the foregoing DNA sequences under standard hybridization conditions; and
(e) DNA sequences that code on expression for an amino acid sequence encoded by any of the foregoing DNA sequences.

The invention thus provides a nucleic acid sequence encoding a mutant human vanilloid polypeptide wherein one or more of amino acids 505, 506, 507, 508, 509, 510, 511, 512, 513, 514, 515, 516 and 517 is replaced with another amino acid.

The invention provides a nucleic acid vector for recombinant expression of a human VR1 receptor which does not bind and/or respond to vanilloid comprising a nucleic acid sequence encoding a human VR1 receptor which does not bind and/or respond to vanilloid, operatively linked to an expression control sequence. The invention includes host cells that have been stably transfected with a nucleic acid vector for recombinant expression of a human VR1 receptor which does not bind and/or respond to vanilloid.

In one aspect, the invention provides an antibody specific to a vanilloid receptor the receptor to which said antibody is raised having the following characteristics:
(a) insensitive to vanilloid;
(b) sensitive to low pH; and
(c) sensitive to heat.

In one aspect, the invention provides an antibody specific to a VR1 receptor the receptor to which said antibody is raised having the following characteristics:
(a) insensitive to vanilloid;
(b) sensitive to low pH; and
(c) sensitive to heat.

The vanilloid-insensitive receptor specific antibody may be a polyclonal antibody or a monoclonal antibody.

The invention includes a method of testing the ability of a drug or other entity to modulate the activity of a vanilloid insensitive receptor which comprises
(a) culturing a colony of test cells which has a vanilloid insensitive receptor in a growth medium containing a known vanilloid receptor agonist or antagonist or in the presence of heat or low pH;
(b) adding the drug under test; and
(c) measuring the reactivity of said known agonist or antagonist with the vanilloid insensitive receptor on said colony of test cells,
   wherein said vanilloid insensitive receptor has the following characteristics:
(i) does not bind or respond to capsaicin
(ii) does respond to low pH;
(iii) does respond to heat.

In a particular embodiment of this method the vanilloid insensitive receptor polypeptide has one or more of amino acids selected from the group of amino acids 505, 506, 507, 508, 509, 510, 511, 512, 513, 514, 515, 516 and 517 replaced with another amino acid.

Yet another aspect of the invention relates to use of mutant vanilloid receptor polypeptides which do not bind and/or do not respond to vanilloid and specific antibodies thereto for the diagnosis and treatment of human disease and painful syndromes. Such diseases and painful syndromes include, but are not limited to pain, chronic pain, neuropathic pain, postoperative pain, rheumatoid arthritic pain, osteoarthritic pain, back pain, visceral pain, cancer pain, algesia, neuralgia, migraine, neuropathies, diabetic neuropathy, sciatica, HIV-related neuropathy, post-herpetic neuralgia, fibromyalgia, nerve injury, ischemia, neurodegeneration and neurodegenerative disorders, stroke, post stroke pain, multiple sclerosis, inflammatory disorders, irritable bowel syndrome, inflammatory bowel disease and burns.

The invention further relates to biologically active agents that modulate vanilloid receptor function, including VR1 receptor and vanilloid receptor-like receptor function, independent of vanilloid binding or response, particularly identified or characterized in the methods of screening provided herein, and their use in the diagnosis and treatment of human disease and painful syndromes. Such diseases and painful syndromes include, but are not limited to pain, chronic pain, neuropathic pain, postoperative pain, rheumatoid arthritic pain, osteoarthritic pain, back pain, visceral pain, cancer pain, algesia, neuralgia, migraine, neuropathies, diabetic neuropathy, sciatica, HIV-related neuropathy, post-herpetic neuralgia, fibromyalgia, nerve injury, ischemia, neurodegeneration and neurodegenerative disorders, stroke, post stroke pain, multiple sclerosis, inflammatory disorders, irritable bowel syndrome, inflammatory bowel disease and burns.

In another aspect the invention features transgenic, non-human mammals expressing a mutant vanilloid receptor-encoding transgene, which does not bind and/or does not respond to vanilloid, and use of such transgenic mammals in screening of candidate vanilloid receptor agonist and antagonist compounds.

The possibilities both diagnostic and therapeutic that are raised by the existence of the vanilloid receptors, derive from the fact that the receptors participate in the response and sensitivity to noxious stimuli, including pain. The advantage of vanilloid insensitive vanilloid receptors is that the response to noxious stimuli, particularly pain or inflammatory stimuli, can be modulated independent of vanilloid responsiveness. Thus specific modulators of the pain response can be screened, identified and isolated. As suggested earlier and elaborated on further herein, the present invention contemplates pharmaceutical intervention in the cascade of reactions in which the vanilloid receptor is implicated, to modulate the activity initiated by the receptor, independent of vanilloid responsiveness.

Thus, in instances where it is desired to reduce or inhibit pain or the response to noxious stimuli, vanilloid receptor modulators could be introduced to block the interaction of the vanilloid receptor with those factors causally connected with the pain or stimulus response, and independent of vanilloid response.

As discussed earlier, the vanilloid-insensitive vanilloid receptors or their binding partners or other ligands, compounds or agents modulating the vanilloid receptors or control over their production, may be prepared in pharmaceutical compositions, with a suitable carrier and at a strength effective for administration by various means to a patient experiencing pain, an inflammatory condition, or other noxious stimulus. A variety of administrative techniques may be utilized, among them parenteral techniques such as subcutaneous, intravenous and intraperitoneal injections, catheterizations and the like. Average quantities of the receptor or receptor modulator or their subunits may vary and in particular should be based upon the recommendations and prescription of a qualified physician or veterinarian.

It is further noted that physical and functional association between different vanilloid receptors, including VR1 and the vanilloid receptor-like receptor VRL3 for instance, has been identified (Davis, J. B. and Smith, G. D., PCT published patent WO 03/074562 A2, incorporated herein by reference in its entirety). Thus, the incorporation of different vanilloid receptor subunits to form homomeric and heteromeric channels, including fully vanilloid insensitive homomeric and heteromeric complexes, is contemplated herein. Isolated heteromeric vanilloid-insensitive proteins are therefore contemplated and provided herein, including comprising an association of VR1 receptor polypeptide and vanilloid receptor-like polypeptide sequences, including wherein one or all of said receptor polypeptide are vanilloid-insensitive or are not vanilloid responsive. Fusion proteins comprising amino acid sequences of vanilloid receptors, including YR1 and vanilloid receptor-like receptor, are further provided herein, including wherein said fusion protein provides a functional, but vanilloid-insensitive or non-responsive channel. The fusion protein may comprise a linker comprising an amino acid sequence other than that contained in the VR1 or vanilloid receptor-like sequence.

Also, antibodies including both polyclonal and monoclonal antibodies, and drugs that modulate the production or activity of the vanilloid-insensitive vanilloid receptor and/or their subunits may possess certain diagnostic applications and may for example, be utilized for the purpose of detecting and/or measuring conditions such as inflammation, pain or noxious stimulus or the like. For example, the vanilloid-insensitive vanilloid receptor or its subunits may be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or antagonize the activity(ies) of the vanilloid-insensitive vanilloid receptor of the invention may be discovered or synthesized, and may be used in diagnostic and/or therapeutic protocols.

The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890.

Panels of monoclonal antibodies produced against vanilloid-insensitive vanilloid receptor peptides can be screened for various properties; i.e., isotype, epitope, affinity, etc. Of particular interest are monoclonal antibodies that neutralize the activity of the vanilloid-insensitive vanilloid receptor or its subunits. Such monoclonals can be readily identified in vanilloid receptor activity assays. High affinity antibodies are also useful when immunoaffinity purification of native or recombinant vanilloid-insensitive vanilloid receptor is possible.

Preferably, the anti-vanilloid receptor antibody used in the diagnostic methods of this invention is an affinity purified polyclonal antibody. More preferably, the antibody is a monoclonal antibody (mAb). In addition, it is preferable for the anti-vanilloid receptor antibody molecules used herein be in the form of Fab, Fab′, F(ab′)₂ or F(v) portions of whole antibody molecules.

Media useful for the preparation of these compositions are both well-known in the art and commercially available and include synthetic culture media, inbred mice and the like. An exemplary synthetic medium is Dulbecco's minimal essential medium (DMEM; Dulbecco et al., Virol. 8:396 (1959)) supplemented with 4.5 gm/l glucose, 20 mm glutamine, and 20% fetal calf serum. An exemplary inbred mouse strain is the Balb/c.

The present invention further contemplates therapeutic compositions useful in practicing the therapeutic methods of this invention. A subject therapeutic composition includes, in admixture, a pharmaceutically acceptable excipient (carrier) and one or more of a vanilloid-insensitive vanilloid receptor, a polypeptide analog thereof or fragment thereof, or a vanilloid-insensitive vanilloid receptor modulator, as described herein as an active ingredient. In a preferred embodiment, the composition comprises an compound capable of modulating the vanilloid-insensitive vanilloid receptor within a target cell.

The preparation of therapeutic compositions which contain compounds, chemical agents, polypeptides, analogs or active fragments as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A compound, agent, polypeptide, analog or active fragment can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic polypeptide-, analog- or active fragment-containing compositions are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of inhibition or neutralization of vanilloid-insensitive vanilloid receptor activity desired. Precise amounts of active ingredient required to be administered depend on the judgment of the practitioner and are peculiar to each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

As used herein, “pg” means picogram, “ng” means nanogram, “ug” or “μg” mean microgram, “mg” means milligram, “ul” or “μl” mean microliter, “ml” means milliliter, “1” means liter.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host.

Such operative linking of a DNA sequence of this invention to an expression control sequence, of course, includes, if not already part of the DNA sequence, the provision of an initiation codon, ATG, in the correct reading frame upstream of the DNA sequence.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

Considering these and other factors a person skilled in the art will be able to construct a variety of vector/expression control sequence/host combinations that will express the DNA sequences of this invention on fermentation or in large scale animal culture.

It is further intended that vanilloid-insensitive vanilloid receptor analogs may be prepared from nucleotide sequences of the protein complex/subunit derived within the scope of the present invention. Analogs, such as fragments, may be produced, for example, by pepsin digestion of vanilloid-insensitive vanilloid receptor material. Other analogs, such as muteins, can be produced by standard site-directed mutagenesis of vanilloid-insensitive vanilloid receptor coding sequences. Analogs exhibiting “vanilloid-insensitive vanilloid receptor activity” such as small molecules, whether functioning as promoters or inhibitors, may be identified by known in vivo and/or in vitro assays.

As mentioned above, a DNA sequence encoding a vanilloid-insensitive vanilloid receptor can be prepared synthetically rather than cloned. The DNA sequence can be designed with the appropriate codons for the vanilloid-insensitive vanilloid receptor amino acid sequence. In general, one will select preferred codons for the intended host if the sequence will be used for expression. The complete sequence is assembled from overlapping oligonucleotides prepared by standard methods and assembled into a complete coding sequence. See, e.g., Edge, Nature, 292:756 (1981); Nambair et al., Science, 223:1299 (1984); Jay et al., J. Biol. Chem., 259:6311 (1984).

Synthetic DNA sequences allow convenient construction of genes which will express vanilloid-insensitive vanilloid receptor analogs or “muteins”. Alternatively, DNA encoding muteins can be made by site-directed mutagenesis of vanilloid-insensitive vanilloid receptor genes or cDNAs, and muteins can be made directly using conventional polypeptide synthesis.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method may be used to create analogs with unnatural amino acids.

The present invention also relates to a variety of diagnostic applications, including methods for detecting the presence of stimuli such as the earlier referenced polypeptide ligands, by reference to their ability to elicit the activities which are mediated by the present vanilloid-insensitive vanilloid receptor.

In these assays or diagnostic applications, one or more component, including the vanilloid-insensitive vanilloid receptor, the vanilloid-insensitive vanilloid receptor antibody or a modulator of the vanilloid-insensitive vanilloid receptor may be labeled. The labels most commonly employed for these studies are radioactive elements, enzymes, chemicals which fluoresce when exposed to ultraviolet light, and others.

A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate. The vanilloid-insensitive vanilloid receptor or its binding partner(s) or modulator can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ⁸⁶Re.

Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

A particular assay system developed and utilized in accordance with the present invention, is known as a receptor assay. In a receptor assay, the material to be assayed is appropriately labeled and then certain cellular test colonies are inoculated with a quantity of both the labeled and unlabeled material after which binding studies are conducted to determine the extent to which the labeled material binds to the cell receptors. In this way, differences in affinity between materials can be ascertained.

Accordingly, a purified quantity of the vanilloid-insensitive vanilloid receptor or the vanilloid-insensitive vanilloid receptor modulator may be radiolabeled and combined, for example, with antibodies or other inhibitors thereto, after which binding studies would be carried out. Solutions would then be prepared that contain various quantities of labeled and unlabeled uncombined vanilloid-insensitive vanilloid receptor or vanilloid-insensitive vanilloid receptor modulator, and cell samples would then be inoculated and thereafter incubated. The resulting cell monolayers are then washed, solubilized and then counted in a gamma counter for a length of time sufficient to yield a standard error of <5%. These data are then subjected to Scatchard analysis after which observations and conclusions regarding material activity can be drawn. While the foregoing is exemplary, it illustrates the manner in which a receptor assay may be performed and utilized, in the instance where the cellular binding ability of the assayed material may serve as a distinguishing characteristic.

An assay useful and contemplated in accordance with the present invention is known as a “cis/trans” assay. Briefly, this assay employs two genetic constructs, one of which is typically a plasmid that continually expresses a particular receptor of interest when transfected into an appropriate cell line, and the second of which is a plasmid that expresses a reporter such as luciferase, under the control of a receptor/ligand complex. Thus, for example, if it is desired to evaluate a compound as a ligand for a particular receptor, one of the plasmids would be a construct that results in expression of the receptor in the chosen cell line, while the second plasmid would possess a promoter linked to the luciferase gene in which the response element to the particular receptor is inserted. If the compound under test is an agonist for the receptor, the ligand will complex with the receptor, and the resulting complex will bind the response element and initiate transcription of the luciferase gene. The resulting chemiluminescence is then measured photometrically, and dose response curves are obtained and compared to those of known ligands. The foregoing protocol is described in detail in U.S. Pat. No. 4,981,784 and PCT International Publication No. WO 88/03168, for which purpose the artisan is referred.

In a further embodiment of this invention, methods for screening modulators of vanilloid-insensitive vanilloid receptor activity, including the prepatration of test kits suitable for use by a medical specialist or skilled artisan, are provided to determine the presence or absence of vanilloid-insensitive vanilloid receptor activity or predetermined vanilloid-insensitive vanilloid receptor modulator activity capability in suspected target cells. In accordance with the methods and testing techniques discussed above and further provided herein, one class of such kits will contain at least the labeled vanilloid-insensitive vanilloid receptor or its binding partner, for instance an antibody specific thereto, and directions, of course, depending upon the method selected, e.g., “competitive,” “sandwich,” “DASP” and the like. The kits may also contain peripheral reagents such as buffers, stabilizers, etc. In as much as the vanilloid-insensitive vanilloid receptor is a receptor mediating a cellular response, including through an alteration in intracellular divalent cations, a test kit may include a means for measuring changes in intracellular divalent cation (for instance Ca²⁺) concentrations or for otherwise measuring receptor activity.

Methods for measuring receptor activity and response are well known and familiar to those of skill in the art and include the use of voltage clamp assays, oocyte assays, and indicator dyes which are sensitive to or record receptor activity and/or changes in intracellular ion concentration. Particular exemplary methods include but are not limited to those provided and described herein, including in the Examples.

Screening for Vanilloid-Insensitive Vanilloid Receptor Modulating Compounds

Vanilloid-insensitive vanilloid receptors or active fragments thereof, can be used for screening compounds that affect vanilloid receptor activity by, for example, specifically binding vanilloid-insensitive vanilloid receptor and affecting its function, thereby affecting vanilloid receptor activity. Identification of such compounds can be accomplished using any of a variety of drug screening techniques. Such compounds or agents are candidates for development of treatments for, inflammatory conditions associated at least in part with vanilloid receptor activity (e.g, psoriasis, reactive airway diseases (e.g., asthma, chronic obstructive pulmonary disease)), arthritis (e.g., osteoarthritis, rheumatoid arthritis), and for use as analgesics. Of particular interest are screening assays for agents that have a low toxicity for human cells. The polypeptide employed in such a test can be free in solution, affixed to a solid support, present on a cell surface, or located intracellularly. The screening assays of the invention are generally based upon the ability of the agent to bind to a vanilloid-insensitive vanilloid receptor polypeptide, and/or elicit or inhibit a vanilloid-insensitive vanilloid receptor-associated activity (i.e., a functional assay or an assay using radioligand binding assays).

Thus, in the assays of the present invention, a vanilloid-insensitive receptor polypeptide is utilized in screening, wherein in a particular embodiment said vanilloid-insensitive vanilloid receptor is lacking a functional vanilloid binding site, particularly wherein it is unable to bind vanilloids. Such vanilloid-insensitivity may be determined, for instance by expression of the vanilloid receptor in oocytes and recording or monitoring currents from voltage-clamped oocytes. These oocytes can be assessed for receptor responsiveness by monitoring current on exposure to heat, by perfusion of acidic solutions, by perfusion with capsaicin or other vanilloid compounds, by perfusion of other receptor active compounds which are not vanilloids, and by exposure to candidate compounds or agents. Failure to evoke a current response on perfusion of an oocyte, particularly a Xenopus oocyte, expressing a vanilloid receptor with capsaicin indicates that the vanilloid receptor is insensitive to vanilloid and/or lacks a functional vanilloid binding site. Jordt and Julius describe methods for determining such responsiveness and vanilloid binding (Jordt S-E and Julius D (2002) Cell 108:421-430).

Particularly, compounds or agents can be screened for agonistic or antagonistic activity in a functional assay that assesses a biological activity associated with vanilloid receptor function, such as effects upon intracellular levels of cations in a vanilloid-insensitive vanilloid receptor expressing host cell (including but not limited to calcium, magnesium, guanidinium, cobalt, potassium, cesium, sodium, and choline, particularly calcium), ligand-activated conductances, cell death (including receptor-mediated cell death which can be monitored using for instance morphological assays, chemical assays or immunological assays), depolarization of the vanilloid-insensitive vanilloid receptor expressing cells (including using fluorescent voltage-sensitive dyes), second messenger production (including through detection of changes in cGMP levels (Wood et al (1989) J Neurochem 53:1203-1211), which can be detected by radioimmunoassay or ELISA), calcium-induced reporter gene expression (for instance Ginty et al (1997) Neuron 18:183-186), or such other readily assayable biological activity associated with vanilloid receptor activity or inhibition of vanilloid receptor activity. Particularly, the functional assay is based upon detection of a biological activity that can be assayed using high-throughput screening of multiple samples simultaneously, for instance an assay based on detection of a change in fluorescence which is associated with a change in vanilloid receptor activity.

The term “agent” or “compound” as used herein describes any molecule, e.g. protein or pharmaceutical, with the capability of altering (i.e., eliciting, stimulating, or inhibiting) or mimicking a desired vanilloid-insensitive vanilloid receptor polypeptide. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts (including extracts from human tissue to identify endogenous factors affecting capsaicin receptor or capsaicin receptor-related polypeptide activity) are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Preferably, the drug screening technique used provides for high throughput screening of compounds having suitable binding affinity to the vanilloid-insensitive vanilloid receptor, and/or eliciting a desired vanilloid-insensitive vanilloid receptor-associated response. For example, large numbers of different small peptide test compounds can be synthesized on a solid substrate, such as plastic pins or some other surface (see, e.g., Geysen WO Application 84/03564, published on Sep. 13, 1984), the peptide test compounds contacted with vanilloid-insensitive vanilloid receptor polypeptides, unreacted materials washed away, and bound vanilloid-insensitive vanilloid receptor detected by virtue of a detectable label or detection of a biological activity associated with vanilloid-insensitive vanilloid receptor activity. Purified vanilloid-insensitive vanilloid receptor polypeptide can also be coated directly onto plates for use in such in vitro drug screening techniques. Alternatively, non-neutralizing antibodies can be used to capture the polypeptide and immobilize it on a solid support.

The invention also contemplates the use of competitive drug screening assays in which vanilloid-insensitive vanilloid receptor-specific neutralizing antibodies compete with a test compound for binding of vanilloid-insensitive vanilloid receptor polypeptide. In this manner, the antibodies can be used to detect the presence of any polypeptide that shares one or more antigenic determinants with a vanilloid-insensitive vanilloid receptor polypeptide.

A wide variety of assays may be used for identification of vanilloid-insensitive vanilloid receptor polypeptide agents, including labeled in vitro binding assays, immunoassays for protein binding, and the like. For example, by providing for the production of large amounts of vanilloid-insensitive vanilloid receptor polypeptides, one can identify ligands or substrates that bind to, modulate or minmic the action of the proteins. The purified protein may also be used for determination of three-dimensional crystal structure, which can be used for modeling intermolecular interactions.

The screening assay can be a binding assay, wherein one or more of the molecules may be joined to a label, and the label directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assays described herein. Where the assay is a binding assay, these include reagents like salts, neutral proteins, e.g. albumin, detergents, etc that are used to facilitate optimal protein-protein binding, protein-DNA binding, and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding.

Preferably, vanilloid-insensitive vanilloid receptor-binding compounds are screened for agonistic or antagonist action in a functional assay that monitors a biological activity associated with vanilloid-insensitive vanilloid receptor function such as effects upon intracellular levels of cations in a vanilloid-insensitive vanilloid receptor-expressing host cell (e.g., calcium, magnesium, guanidinium, cobalt, potassium, cesium, sodium, and choline, preferably calcium), ligand-activated conductances, cell death (i.e., receptor-mediated cell death which can be monitored using, e.g., morphological assays, chemical assays, or immunological assays), depolarization of the vanilloid-insensitive vanilloid receptor-expressing cells (e.g., using fluorescent voltage-sensitive dyes), second messenger production (e.g., through detection of changes in cyclic GMP levels (see, e.g., Wood et al. 1989 J. Neurochem. 53:1203-1211), which can be detected by radioimmunoassay or ELISA), calcium-induced reporter gene expression (see, e.g., Ginty 1997 Neuron 18:183-186), or other readily assayable biological activity associated with vanilloid receptor activity or inhibition of vanilloid receptor activity. Preferably, the functional assay is based upon detection of a biological activity of vanilloid receptor that can be assayed using high-throughput screening of multiple samples simultaneously, e.g., a functional assay based upon detection of a change in fluorescence which in turn is associated with a change in vanilloid receptor activity. Such functional assays can be used to screen candidate agents for activity as either vanilloid-insensitive vanilloid receptor agonists or antagonists.

In a preferred embodiment, vanilloid-insensitive vanilloid receptor-expressing cells (preferably recombinant vanilloid-insensitive vanilloid receptor-expressing cells) are pre-loaded with fluorescently-labeled calcium (e.g, fura-2). The capsaicin receptor-expressing cells are then exposed to a candidate vanilloid-insensitive vanilloid receptor-modulating compound and the effect of exposure to the compound monitored. Candidate compounds that have vanilloid-insensitive vanilloid receptor agonist activity are those that, when contacted with the vanilloid-insensitive vanilloid receptor-expressing cells, elicit a vanilloid receptor-mediated increase in intracellular calcium relative to control cells (e.g., vanilloid-insensitive vanilloid receptor-expressing cells in the absence of the candidate compound, host cells without vanilloid-insensitive vanilloid receptor-encoding nucleic acid, vanilloid-insensitive vanilloid receptor-expressing cells exposed to a known vanilloid receptor agonist). Similarly, functional vanilloid receptor assays can be used to identify candidate compounds that block activity of a known vanilloid receptor agonist (e.g., block the activity of or compete with resiniferatoxin), block activity of a known vanilloid receptor antagonist (e.g., block the activity of or compete with BCTC), and/or have activity as vanilloid receptor antagonists.

Accordingly, a kit may be prepared for the demonstration of the presence or capability of cells for predetermined vanilloid-insensitive vanilloid receptor activity, comprising:
(a) a predetermined amount of at least one of:
   (i) a labeled immunochemically reactive component obtained by the direct or indirect attachment of the vanilloid-insensitive vanilloid receptor or a specific binding partner thereto, to a detectable label; or
   (ii) a fluorescent or other indicator which indicates the amount or changes in the intracellular concentration of ions;
(b) other reagents; and
(c) directions for use of said kit.

In a further variation, the test kit may be prepared and used for the purposes stated above, which operates according to a predetermined protocol (e.g. “competitive,” “sandwich,” “double antibody,” etc.), and comprises:
(a) a labeled component which has been obtained by coupling the vanilloid-insensitive vanilloid receptor to a detectable label;
(b) one or more additional immunochemical reagents of which at least one reagent is a ligand or an immobilized ligand, which ligand is selected from the group consisting of:
   (i) a ligand capable of binding with the labeled component (a);
   (ii) a ligand capable of binding with a binding partner of the labeled component (a);
   (iii) a ligand capable of binding with at least one of the component(s) to be determined; and
   (iv) a ligand capable of binding with at least one of the binding partners of at least one of the component(s) to be determined; and
(c) directions for the performance of a protocol for the detection and/or determination of one or more components of an immunochemical reaction between the vanilloid-insensitive vanilloid receptor and a specific binding partner thereto.

In accordance with the above, an assay system for screening potential drugs effective to modulate the activity of the vanilloid-insensitive vanilloid receptor may be prepared. The vanilloid-insensitive vanilloid receptor may be introduced into a test system, and the prospective drug may also be introduced into the resulting cell culture, and the culture thereafter examined to observe any changes in the vanilloid-insensitive vanilloid receptor activity of the cells, due either to the addition of the prospective drug alone, or due to the effect of added quantities of the known vanilloid-insensitive vanilloid receptor.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLE 1

The human vanilloid receptor (VR1) is a ligand-gated ion channel activated by various endogenous agonists, vanilloids, acid, and heat, thus acting as a molecular detector and integrator of multiple modes of pain. These receptors are expressed in a specific subset of pain-sensing DRG neurons and are involved in certain pathological forms of pain. Finding specific blockers of this receptor may result in the discovery of novel analgesic drugs.

Current strategies used in the industry for the discovery of VR1 antagonists are based on capsaicin-displacement screens. These drugs prevent capsaicin from interacting with the receptor and therefore block capsaicin-induced activation of the channel. However, blocking the channel in this way does not necessarily prevent endogenous activators (anandamide, acid, heat, etc.) from activating VR1. Here we present a method to screen for vanilloid receptor, including VR1, agonists and antagonists that specifically interact with the channel independent of the capsaicin-binding site or a functioning capsaicin response. In one embodiment, this is achieved by screening drugs against an altered form of the VR1 receptor that has a disrupted capsaicin binding site. Therefore, drugs that block the receptor by interacting with the capsaicin binding site are excluded, allowing the discovery of drugs that modulate the receptor independent of a functional capsaicin binding site that may be more physiologically relevant.

Primary sensory neurons from chicks are capsaicin insensitive, but do exhibit heat-evoked membrane currents suggesting that birds may express a vanilloid-insensitive homolog of VR1 (Wood, J. N. et al (1998) J Neurosci 8:3208-3220; Marin-Burgin, A. et al (2000) Eur J Neurosci 12:3560-3566; Nagy, I. and Rang, H. (2000) Regul Pept 96:3-6). Jordt and Julius have studied the molecular basis for species-specific sensitivity to hot chili peppers, cloning the avian vanilloid receptor ortholog and comparing its sequence to that of rat VR1 (Jordt and Julius (2002) Cell 108:421-430). This sequence comparison identified structural determinants for vanilloid interaction in a transmembrane segment of the rat receptor. More recently, McIntyre et al have studied and reported pharmacological differences between human and rat VR1 (McIntyre et al (2001) Br J Pharmacol 132:1084-1094). Although capsaicin had similar potency at the two receptors, rat and human VR1 responded differently to the non-pungent RTX analogue phorbol 12-phenylacetate 13 acetate 20-homovanillate (PPAHV), which was essentially inactive up to a concentration of 10•M on human VR1 (EC₅₀ values in rat between 3 and 10•M). In addition, capsazine, a competitive capsaicin antagonist (Bevan et al (1992) Br J Pharmacol 107:544-552), showed species-dependent inhibitory effects, with approximately 6 fold greater potency of inhibition of capsaicin activation on human VR1 receptors than rat. Capsazine also blocked the low pH-evoked responses of human but not rat VR1 and was highly effective at blocking the human-VR1 mediated heat response, while only a weak inhibitor of the heat response of rat VR1. These data suggest that the site and mechanism of action of interaction/binding with and activation of VR1 by capsaicin is different from that of protons and heat, and that these responses differ between rats and humans.

Results

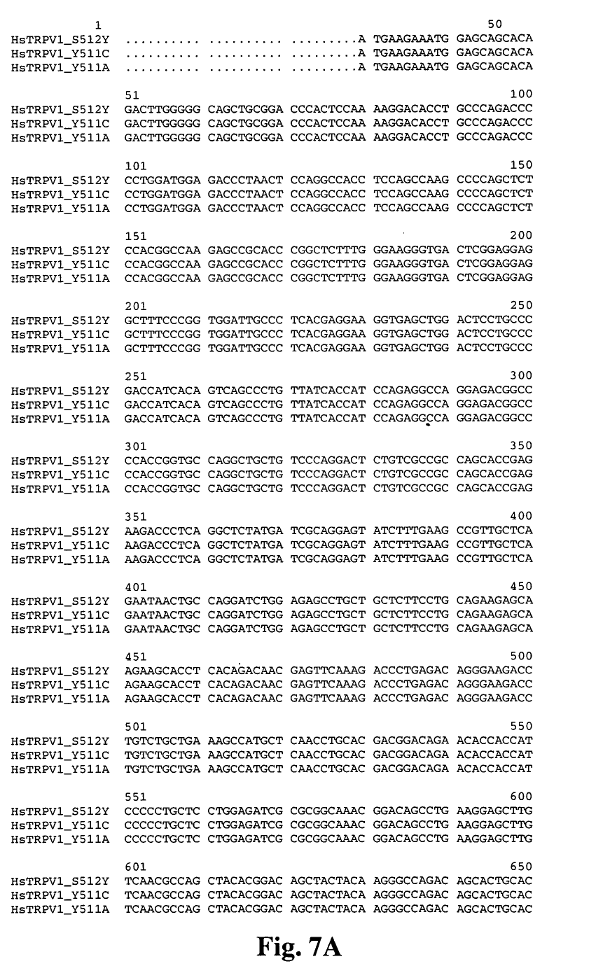

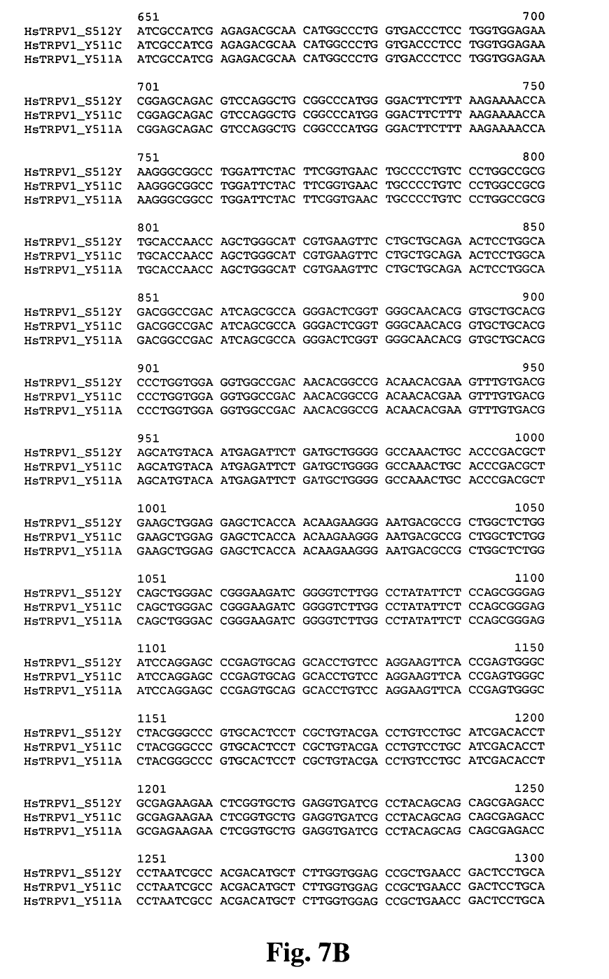

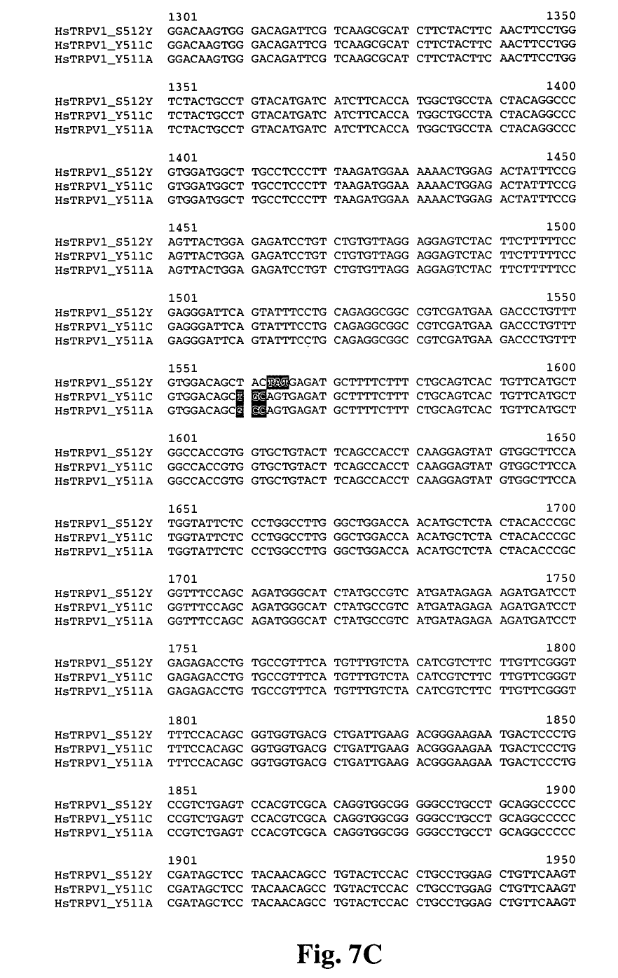

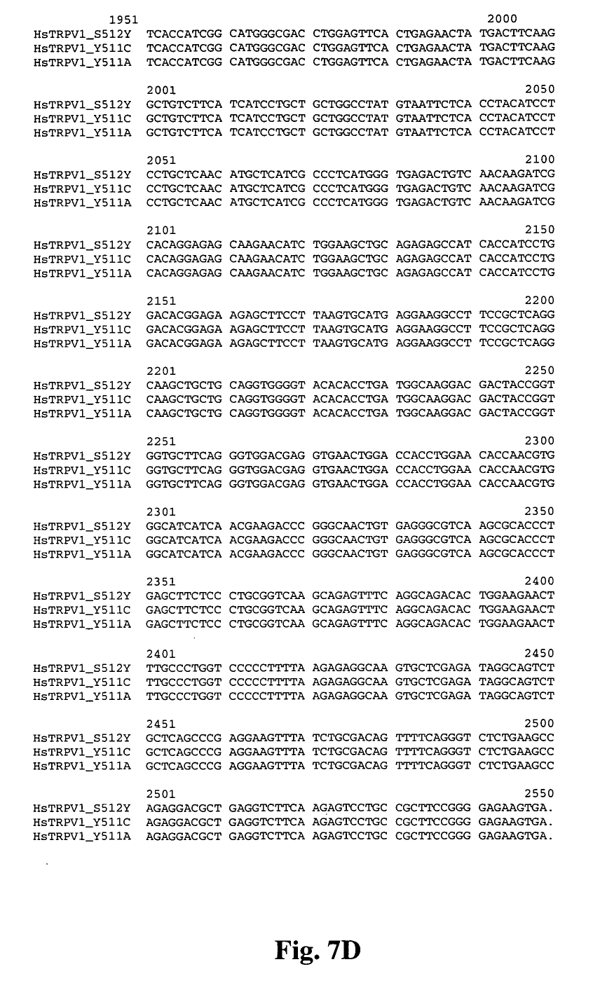

Three initial specific mutant VR1 constructs were generated in the human VR1 channel, utilizing the unique human VR1 of FIG. 8. Two of the constructs have a mutation at amino acid position 511 and have been mutated from the amino acid tyrosine to either alanine or cysteine (we refer to these constructs as Y511A and Y511C, respectively). The third construct has a single point mutation at amino acid position 512 where we have mutated the residue from serine to tyrosine (we refer to this construct at S512Y). The full DNA sequence and predicted protein sequence of these mutant VR1 open reading frames are shown in FIGS. 1, 2, and 3 for constructs Y511A, Y511C, and S512Y respectively. A comparison of the nucleic acid sequences of these mutant VR1 receptors is shown in FIG. 7.

The mutations were generated using PCR mutagenesis techniques. Briefly, DNA oligonucleotides containing each mutation were used to amplify a region of human VR1 DNA flanked by BclII restriction sites. The products of this PCR amplification were digested with BclII and inserted back into the original construct (also cut with BclII). The resulting DNA constructs were sequenced to verify the presence of the mutations as well as to determine whether any additional mutations were inadvertently introduced.

The resulting sequence-verified mutant VR1 DNA was inserted into the pcDNA3.1 vector (Invitrogen). Human HEK293 cells were then transfected with the DNA, incubated at 37° C. for 24-48 hrs, and then analyzed for receptor response using calcium imaging techniques.

Calcium imaging was performed using a Zeiss Axiovert 200M fluorescence microscope equipped with a Photonics Polychrome IV monochrometer fluorescence excitation system as well as a multivalve perfusion system for delivering drugs. Cells were plated on 12 mm glass coverslips and loaded with fura2-AM for 30 min at 37° C. Imaging was performed by alternatively measuring the fluorescence excited at 340 nm and 380 nm at 5 sec time intervals. The ratio between these measured fluorescence values (F340/F380) was then calculated and graphed as a function of time. The 340/380 fluorescence ratio is proportional in a nonlinear way to the intracellular free calcium concentration. Since VR1 is a calcium channel, its opening results in an influx of calcium into the cytoplasm and an increase in the 340/380 fluorescence ratio.

The mutant channels Y511A, Y511C, and S512Y have also been subcloned into the vector pcDNA5/TO (Invitrogen). This expression vector allows regulated protein expression in mammalian cells. Thus, the VR1 mutant channels will only be expressed when tetracycline is added to the growth medium. This allows the stable expression of the mutant VR1 constructs in 293 cells. These cells are used to set up a fluorescence-based high-throughput screening assay using the Molecular Devices Flexstation platform.

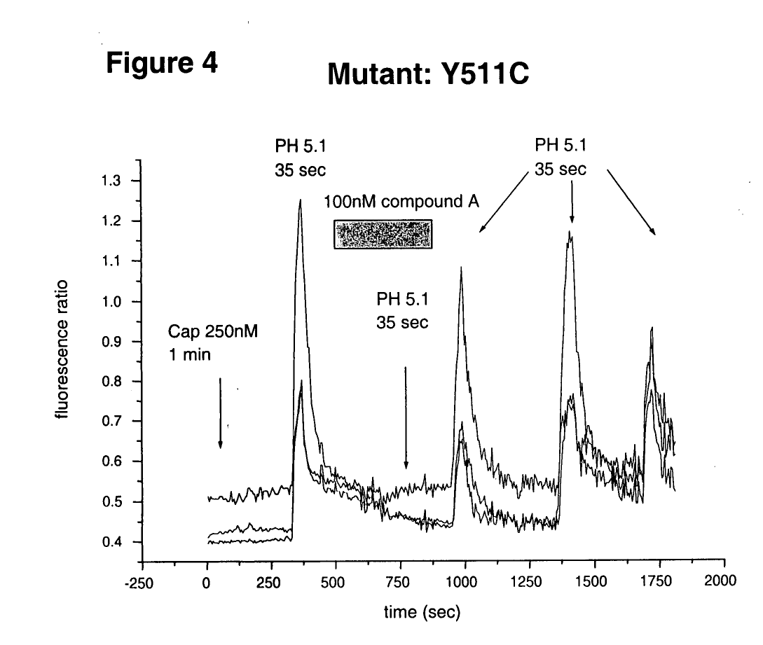

FIG. 4 shows typical results obtained from the mutant VR1 receptor Y511C using calcium imaging with fura-2. A normal saline (pH 7.6) wash solution was used as the control solution. Under these conditions, a low pH saline solution (pH 5.1) causes a large influx of calcium into the transfected 293 cells as a result of VR1 activation. These mutant receptors have no detectable response to 250 nM capsaicin and are thus capsaicin-insensitive channels. 100 nM Compound A (a benzamide derivative and a putative VR1 receptor antagonist) is shown to completely block the effect of low pH on these channels. Compound A has an IC₅₀ of 90 nM for channel blocking activity. Thus, this drug can block VR1 even when the capsaicin binding site has been completely disrupted. We have also verified that the mutant channels Y511A and S512Y similarly make acid-sensitive and capsaicin-insensitive channels that can also be at least partially blocked by Compound A. A related compound, Compound 18, also blocks the mutant VR1 receptor channel, with an IC₅₀ of 135 nM for channel blocking activity. Compound 18 is named 4-(3-Chloropyridin-2-yl)-N-(4-trifluoromethyl-phenyl)benzamide, and is described and disclosed in commonly owned co-pending International Application No. PCT/US04/033099 filed Oct. 7, 2004, which is incorporated herein by reference in its entirety.

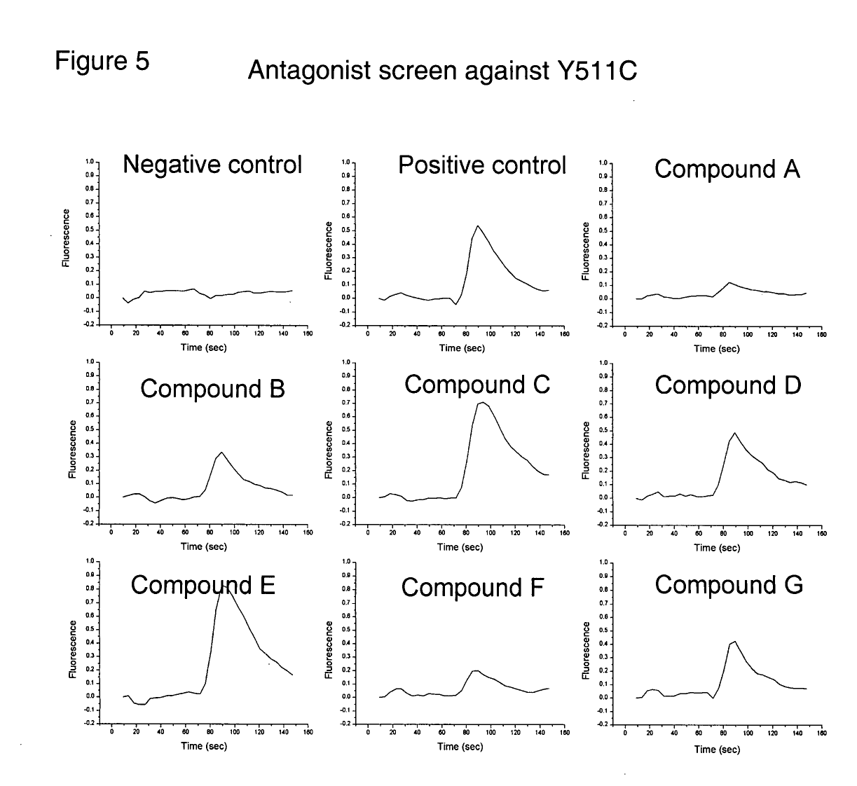

A high-throughput screen utilizing ratiometric fluorimetry using a Molecular Devices Flexstation was set up and results depicted in FIG. 5. The mutant Y511C VR1 receptor is expressed stably in HEK 293 cells, loaded with fura-2, and analyzed by measuring the ratio of fluorescence excited by 340 nm and 380 nm. Shown in FIG. 5 are plots of the fluorescence ratio versus time. The control plots demonstrate the effects of recording the cells either with (positive control) or without (negative control) a low pH (pH 5.1) stimulus to open the channels. The compound plots show the effect of a 300 nM concentration of each test compound, Compound A through G, added 20 sec. before the addition of the low pH stimulus. Compound A almost completely blocks the mutant channel, while the other compounds tested in this screen show varying amounts of block. Screens of this type can be used to identify high affinity antagonists of the mutant VR1 receptor.

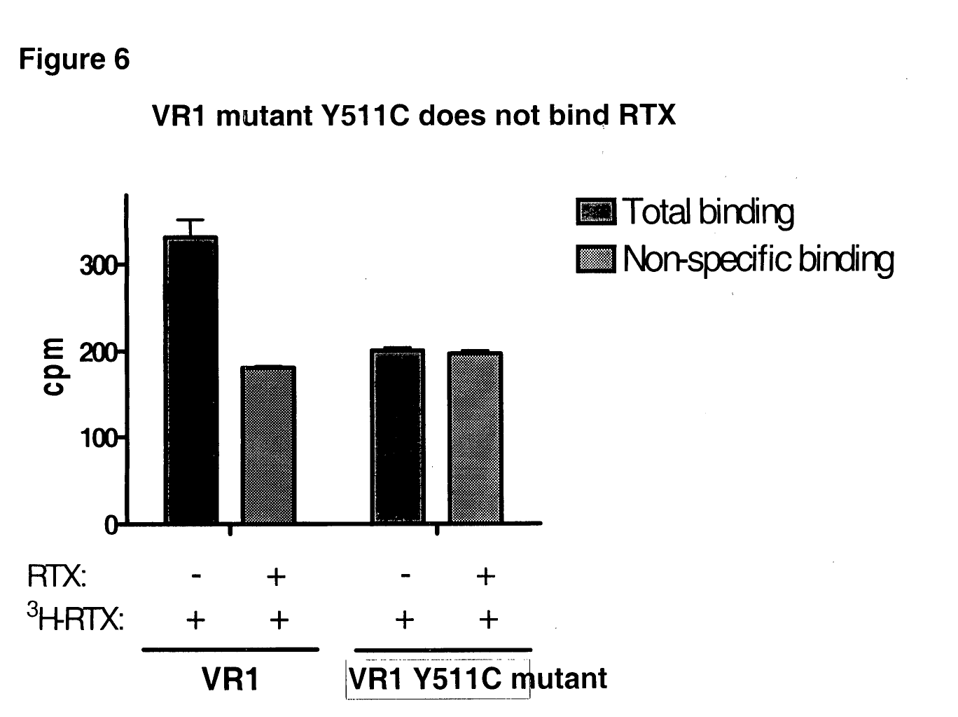

Specific binding of ³H-Resiniferatoxin (RTX) to membranes of 293 HEK cells expressing human VR 1 and mutant VR 1 receptor Y511C was assessed and the results are presented in FIG. 6. Binding of ³H-RTX (0.5 nM) to the membranes was conducted in the absence (labeled total binding) or presence (labeled non specific binding) of unlabeled RTX (200 nM) as indicated. The results demonstrate that RTX shows no significant binding to the mutant VR1 receptor in this concentration range.

We have created three initial mutant VR1 constructs that can be used for high-throughput screening of modulators of the VR1 receptor. We have shown that these mutant channels are not longer activated by capsaicin, but are still blocked by certain VR1 antagonist compounds that interact with vanilloid insensitive VR1 receptor, independent of a functional capsaicin binding site.

Additional vanilloid-insensitiveVR1 mutant constructs are generated utilizing the PCR mutagenesis strategy described above. Mutants are generated at one or more of amino acids 505, 506, 507, 508, 509, 510, 511, 512, 513, 514, 515, 516 and 517 as follows (mutant nomenclature is as noted above, ie., T505A is a mutant wherein a tyrosine at amino acid 505 is replaced by alanine):
T505A
L506A
F507A
V508A
V508F
D509R
D509K
S510Y
S510G
S510E
S510R
S510W
Y511S
Y511V
Y511G
Y511L
Y511I
Y511T
Y511M
S512G
E513A
E513R
E513V
E513C
M514A
M514E
M514R
M514C
M514F
L515A
F516A
F517A

VR1 vanilloid-insensitive mutants having more than one mutation are also generated as follows:
Y511V+S512A
Y511V+S512V
Y511V+S512L
Y511S+S512A
Y511S+S512V
Y511S+S512L

### EXAMPLE 2

Cells expressing VR1 mutant receptors which are vanilloid- or capsaicin-insensitive are assayed for cellular response in the presence and absence of VR1 modulators and test compounds. Examples of representative assays include:

DRG Electrophysiology VR1 Assay

DRG neurons are recovered from either neonatal or adult mice (C57B16). These neurons are plated onto poly-D-lysine coated glass coverslips and placed into a perfusion chamber. This chamber allows drug solutions to be added to the cells using a computer-controlled solenoid-valve based perfusion system. The cells are imaged using standard DIC optics. Cells are patched using finely-pulled glass electrodes containing an intracellular solution consisting of. Voltage-clamp electrophysiology experiments are carried out using an Axon Instruments Multiclamp amplified controlled by pCLAMP8 software. The cells are placed into whole-cell voltage-clamp and help at a voltage of −80 mV while monitoring the membrane current in gap-free recording mode. 500 nM capsaicin is added for 30 sec as a control. Drugs at various concentrations are added to the cells for 1 min prior to a 30 sec capsaicin application. Differences between control experiments and drug+capsaicin experiments are used to determine the efficacy of each drug.

High-Throughput Xenopus oocyte Two-Electrode Voltage Clamp Assay

Capsaicin-insensitive VR1 variants are expressed in Xenopus oocytes and assayed using two-electrode voltage-clamp. Capped RNA for each VR1 variant is synthesized in-vitro using T7 RNA polymerase. RNA is then injected into Xenopus oocytes, which are then allowed to recover for 2-4 days prior to the experiment. Injected oocytes are placed 8 at a time into an Axon Instruments OpusExpress TEVC system for analysis. This instrument automatically inserts current-injection and voltage-recording electrodes into the oocyte and adds drug solutions to the oocytes using a robotic fluid handling system. Oocytes are voltage-clamped and help at −80 mV. A low pH agonist solution (pH 5.1) is added to the oocytes as a control for channel expression. Drug solutions are then added for 1 min prior to a 30 sec application of the low pH solution. Differences between the control experiment and the drug+low pH experiment are used to determine the efficacy of each drug.

High-Throughput Screening for VR1 Antagonists

The functional activity of compounds at the VR1 receptor was determined by measuring intracellular Ca²⁺ levels. A dual wavelength ratiometric dye, Fura2, was used as an indicator of relative levels of [Ca²⁺] in a 96 well format using a bench top scanning fluorometer with integrated fluidics and temperature control (Flex Station, Molecular Devices).

293 cells expressing capsaicin-insensitive VR1 receptor variants were grown to confluency on PDL coated 96-well black-walled plates, in the presence of DMEM medium containing 5% Penstrep, 5% Glutamax, 200 ug/mL Hygromycin, 5 ug/mL Blasticide and 10% heat inactivated FBS. Priory to assay, cells were loaded with 5 ug/mL Fura2 in normal saline solution at 37° C. for 40 minutes. Cells were then washed with normal saline to remove dye before commencement of assay in Flex Station.

The assay consists of two stages; a pre-treatment phase followed by a treatment phase. First, 50 ul of compound solution is added to the cells 20 secs into the experimental run (Pre-treatment). Immediately following, 50 ul of VR1 agonist (saline solution of low pH 5.1) is added together with 50 ul of compound solution (Treatment).

Total time for experimental run was 3 minutes and changes in wavelength measurements were made throughout out the course of the experiment at 4 second intervals. Responses are measured as peak fluorescent ratio after compound-agonist addition minus baseline fluorescent ratio prior to pre-treatment and are calculated using the SoftMaxPro software. Data is expressed as percentage inhibition calculated as follows using Excel.

$$\text{Percentage Inhibition} = \frac{\begin{matrix}\text{(Compound Response)} - \\ \text{(Control Response)}\end{matrix}}{\begin{matrix}\text{(Agonist Response} - \\ \text{Control Response)}\end{matrix}} \times 100$$

All compounds with percentage inhibition values greater than 75% are considered hits and earmarked for further investigation at lower concentrations.

### EXAMPLE 3

The ability of capsaicin to activate the mutant VR1 receptors was evaluated in an oocyte system. cDNAs containing the coding sequences of the unmutated human VR1 receptor as well as the mutant receptors Y511A, Y51C, and S512Y were placed into the multiple-cloning site in the pTNT vector (Promega), which contains a T7 promoter sequence, the beta-globin 5′UTR, and a polyadenylation sequence to enhance translation and RNA stability. RNA was synthesized in-vitro using T7 RNA polymerase. The RNA for each channel was injected into Xenopus oocytes (obtained from Nasco, Inc) and incubated at 16° C. for 3-5 days. Standard two-electrode voltage clamp recordings were used to analyze the function of each expressed channel. Solutions were passed over the oocytes using a multiple-valve perfusion system. FIG. 9 shows results demonstrating that capsaicin fails to activate the mutant VR1 receptors Y511A and Y511C. A typical recording of the wild-type human VR1 receptor showing the large response elicited by the application of 10 μM capsaicin is shown in FIG. 9A. The mutant VR1 receptors Y511C (FIG. 9B) and Y511A (FIG. 9C) demonstrate no detectable capsaicin response even at capsaicin concentrations as high as 100 μM. The mutant receptor S512Y shows a small capsaicin response at 100 μM capsaicin (FIG. 9D) but no detectable response at 10 μM capsaicin (data not shown).

This invention may be embodied in other forms or carried out in other ways without departing from the spirit or essential characteristics thereof. The present disclosure is therefore to be considered as in all aspects illustrate and not restrictive, the scope of the invention being indicated by the appended claims, and all changes which come within the meaning and range of equivalency are intended to be embraced therein.

Various references are cited throughout this Specification, each of which is incorporated herein by reference in its entirety.

1. A method for detecting a compound that modulates the VR1 receptor, comprising:
(a) contacting a sample suspected of containing a VR1 receptor modulating compound with a host cell expressing a VR1 receptor which is insensitive to vanilloid; and
(b) detecting an alteration of a cellular response associated with VR1 receptor activity in the VR1 receptor expressing cost cell.

2. A method for detecting a compound that modulates the VR1 receptor, wherein said compound binds or otherwise interacts with the VR1 receptor independent of the vanilloid-binding site, comprising:
(a) contacting a sample suspected of containing a VR1 receptor modulating agent/compound with a host cell expressing a VR1 receptor which does not bind vanilloid compounds; and
(b) detecting a alteration of a cellular response associated with VR1 receptor activity in the VR1 receptor expressing cost cell.

3. The method of claim 1 or 2 wherein the cellular response associated with VR1 receptor activity is an increase in intracellular calcium concentration.

4. A method for screening for biologically active agents that modulate vanilloid receptor function independent of vanilloid binding or response comprising:
(a) combining a candidate agent with any one of:
(i) a mammalian VR1 receptor polypeptide which does not bind and/or respond to vanilloid compounds;
(ii) a mammalian vanilloid receptor-like polypeptide which does not bind and/or respond to vanilloid compounds;
(iii) a cell containing a nucleic acid encoding a mammalian VR1 receptor polypeptide which does not bind and/or respond to vanilloid compounds;
(iv) a cell containing a nucleic acid encoding a mammalian vanilloid receptor-like polypeptide which does not bind and/or respond to vanilloid compounds; and
(b) determining the effect of said agent on VR1 receptor or vanilloid receptor-like receptor function.

5. An isolated human VR1 receptor polypeptide wherein the polypeptide is insensitive to vanilloid.

6. The VR1 receptor polypeptide of claim 5 wherein one or more amino acids in the vanilloid binding site are altered such that the VR1 receptor does not bind to and/or respond to vanilloid compounds.

7. The VR1 receptor polypeptide of claim 5 or 6 which does not bind to and/or respond to capsaicin.

8. The VR1 receptor polypeptide of claim 5 or 6 wherein one or more of amino acids selected from the group of amino acids 505, 506, 507, 508, 509, 510, 511, 512, 513, 514, 515, 516 and 517 is replaced with another amino acid.

9. The VR1 receptor polypeptide of claim 8 wherein one or more of amino acids is replaced with a non-conservative amino acid.

10. An isolated polynucleotide encoding a human VR1 polypeptide of claim 5.

11. A nucleic acid vector for recombinant expression of a human VR1 receptor which is insensitive to vanilloid comprising a nucleic acid sequence encoding the polypeptide of claim 5, operatively linked to an expression control sequence.

12. A cell that has been stably transfected with the nucleic acid vector of claim 11.

13. A DNA sequence, which encodes a vanilloid insensitive VR1 receptor, or a fragment thereof having receptor activity, selected from the group consisting of:
(a) the DNA sequence of FIG. 1 (SEQ ID NO:);
(b) the DNA sequence of FIG. 2 (SEQ ID NO:);
(c) the DNA sequence of FIG. 3 (SEQ ID NO:);
(d) DNA sequences that hybridize to any of the foregoing DNA sequences under standard hybridization conditions; and
(e) DNA sequences that code on expression for an amino acid sequence encoded by any of the foregoing DNA sequences.

14. An antibody specific to a vanilloid receptor the receptor to which said antibody is raised having the following characteristics:
(a) insensitive to vanilloid;
(b) sensitive to low pH; and
(c) sensitive to heat.

15. An antibody specific to a VR1 receptor the receptor to which said antibody is raised having the following characteristics:
(a) insensitive to vanilloid;
(b) sensitive to low pH; and
(c) sensitive to heat.

16. The antibody of claim 14 or 15 which is a polyclonal antibody.

17. The antibody of claim 14 or 15 which is a monoclonal antibody.

18. An immortal cell line that produces a monoclonal antibody according to claim 17.

19. The antibody of claim 14 or 15 labeled with a detectable label.

20. The antibody of claim 19 wherein the label is selected from enzymes, chemicals which fluoresce and radioactive elements.

21. A method of testing the ability of a drug or other entity to modulate the activity of a vanilloid insensitive receptor which comprises
(a) culturing a colony of test cells which has a vanilloid insensitive receptor in a growth medium containing a known vanilloid receptor agonist or antagonist or in the presence of heat or low pH;
(b) adding the drug under test; and
(c) measuring the reactivity of said known agonist or antagonist with the vanilloid insensitive receptor on said colony of test cells,
wherein said vanilloid insensitive receptor has the following characteristics:
(i) does not bind or respond to capsaicin
(ii) does respond to low pH;
(iii) does respond to heat.

22. The method of claim 21 wherein said vanilloid insensitive receptor polypeptide has one or more of amino acids selected from the group of amino acids 505, 506, 507, 508, 509, 510, 511, 512, 513, 514, 515, 516 and 517 replaced with another amino acid.

23. An isolated heteromeric vanilloid-insensitive protein comprising an association of VR1 receptor polypeptide and a vanilloid receptor-like polypeptide sequences, wherein one or all of said receptor polypeptides are vanilloid-insensitive or are not responsive to vanilloid.

24. The isolated heteromeric vanilloid-insensitive protein of claim 23 wherein the VR1 polypeptide is a human VR1 wherein one or more amino acids in the vanilloid binding site are altered such that the VR1 receptor does not bind to and/or respond to vanilloid compounds.

25. The isolated heteromeric vanilloid-insensitive protein of claim 24 wherein one or more of amino acids selected from the group of amino acids 505, 506, 507, 508, 509, 510, 511, 512, 513, 514, 515, 516 and 517 is replaced with another amino acid.

26. An isolated VR1 receptor polypeptide comprising the amino acid sequence as set out in FIG. 8.

27. An isolated polynucleotide encoding the VR1 polypeptide of claim 26.
